(19) United States
(12) Patent Application Publication
(10) Pub. No.: US 2009/0197867 A1
(43) Pub. Date: Aug. 6, 2009

(54) ENANTIOMERICALLY ENRICHED IMIDAZOAZEPINONE COMPOUNDS

(76) Inventors: Mark Spyvee, Hampstead, NH (US); Christina J. Shaffer, Litchfield, NH (US); Boris M. Seletsky, Andover, MA (US); Shawn Schiller, Haverhill, MA (US); Jia Liu, West Chester, PA (US); Xiang-Yi Li, Andover, MA (US); Qian Chen, Andover, MA (US); Roch Boivin, Lawrence, MA (US)

Correspondence Address: MYERS BIGEL SIBLEY & SAJOVEC, PO BOX 37428, RALEIGH, NC, 27627, US

(21) Appl. No.: 12/270,892

(22) Filed: Nov. 14, 2008

Related U.S. Application Data
(60) Provisional application No. 60/988,247, filed on Nov. 15, 2007.

(51) Int. Cl. A61K 31/55; C07D 487/10; A61P 37/06

(52) U.S. Cl. 514/212.02; 540/543

(57) ABSTRACT

The present invention provides an enantiomerically pure compound of Formula I:

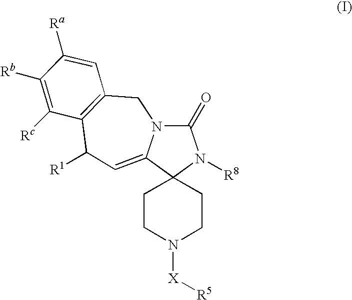

along with pharmaceutical formulations containing the same and methods of use thereof.

### RELATED APPLICATIONS

This application claims the benefit of U.S. Provisional Application No. 60/988,247, filed Nov. 15, 2007, the disclosure of which is incorporated by reference herein in its entirety.

### BACKGROUND OF THE INVENTION

Upon encountering antigen, naive CD4+ T helper precursor (Thp) cells are differentiated into two distinct subsets, Type 1 T helper (Th1) and Type 2 T helper (Th2). These differentiated Th cells are defined both by their distinct functional abilities and by unique cytokine profiles. Specifically, Th1 cells produce interferon-gamma, interleukin (IL)-2, and tumor necrosis factor (TNF)-beta, which activate macrophages and are responsible for cell-mediated immunity and phagocyte-dependent protective responses. In contrast, Th2 cells are known to produce IL-4, IL-5, IL-6, IL-9, IL-10 and IL-13, which are responsible for strong antibody production, eosinophil activation, and inhibition of several macrophage functions, thus providing phagocyte-independent protective responses. Accordingly, Th1 and Th2 cells are associated with different immunopathological responses.

In addition, the development of each type of Th cell is mediated by a different cytokine pathway. Specifically, it has been shown that IL-4 promotes Th2 differentiation and simultaneously blocks Th1 development. In contrast, IL-12, IL-18 and IFN-gamma are the cytokines critical for the development of Th1 cells. Accordingly, the cytokines themselves form a positive and negative feedback system that drives Th polarization and keeps a balance between Th1 and Th2.

Th1 cells are involved in the pathogenesis of a variety of organ-specific autoimmune disorders, Crohn's disease, Helicobacter pylori-induced peptic ulcer, acute kidney allograft rejection, and unexplained recurrent abortions. In contrast, allergen-specific Th2 responses are responsible for atopic disorders in genetically susceptible individuals. Moreover, Th2 responses against still unknown antigens predominate in Omenn's syndrome, idiopathic pulmonary fibrosis, and progressive systemic sclerosis.

There remains a high unmet medical need to develop new therapeutic treatments that are useful in treating the various conditions associated with imbalanced Th1/Th2 cellular differentiation. For many of these conditions the currently available treatment options are inadequate. Accordingly, the Th1/Th2 paradigm provides a rationale for the development of strategies for the therapy of allergic and autoimmune disorders.

### SUMMARY OF THE INVENTION

A first aspect of the present invention is an enantiomerically pure compound (sometimes referred to as an “active compound” herein) of Formula I:

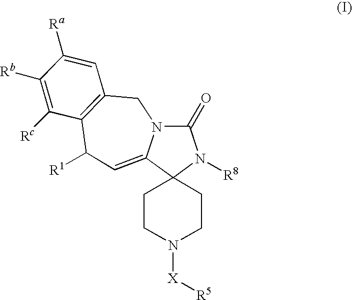

or more particularly Formula Ia or Formula Ib:

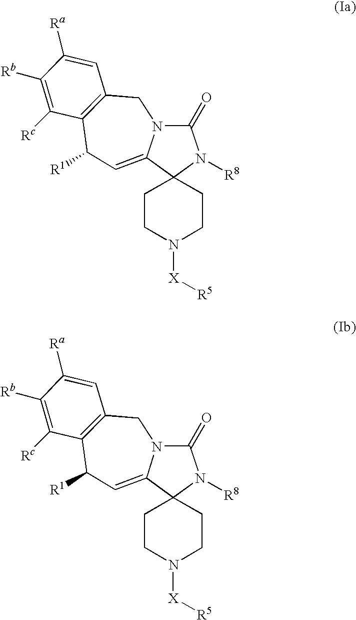

wherein:

R¹ is C₁₋₃ alkyl;

X is methylene, ethylene, propylene, ethenylene, propenylene, or butenylene;

R⁵ is phenyl, pyrrolyl, benzimidazolyl, oxazolyl, isoxazolyl, imidazothiazolyl, quinolinyl, isoquinolinyl, indazolyl, pyridinyl, imidazopyridinyl, indolyl, benzotriazolyl, imidazolyl, benzofuranyl, benzothiadiazolyl, pyridimidinyl, benzopyranonyl, thiazolyl, thiadiazolyl, furyl, thienyl, pyrazolyl, quinoxalinyl, or naphthyl, and substituted with between 0 and 5 substituents independently selected from C₁₋₄ alkyl, C₁₋₃ alkoxy, hydroxyl, C₁₋₃ alkylthio, cyclopropyl, cyclopropylmethyl, trifluoromethoxy, 5-methylisoxazolyl, pyrazolyl, benzyloxy, acetyl, (cyanyl)C₁₋₃ alkyl, (phenyl)C₂₋₃ alkenyl; and halo; R⁸ is H, methyl, ethyl, propyl, (C₁₋₃ alkoxy)C₁₋₃ alkyl, (C₁₋₃ alkylthio)C₁₋₃ alkyl, C₁₋₃ hydroxyalkyl, phenyl, benzyl, furyl, pyrrolyl, imidazolyl, pyrazolyl, pyrrolyl, isothiazolyl, isooxazolyl, pyridyl, and thienyl;

wherein R⁸ is substituted with between 0 and 3 substituents independently selected from methyl, ethyl, halo, hydroxyl, C₁₋₃ alkoxy, C₁₋₃ alkylthio, (C₁₋₃ alkoxy)C₁₋₃ alkyl, (C₁₋₃ alkylthio)C₁₋₃ alkyl, C₁-3 hydroxyalkyl, (C₁₋₃ mercaptoalkyl)phenyl, benzyl, furyl, imidazolyl, pyrazolyl, pyrrolyl, isothiazolyl, isooxazolyl, pyridyl, and thienyl; and

each of R^{a}, R^{b}, and R^{c} is independently selected from hydrogen, hydroxyl, methoxy, benzyloxy, fluoro, chloro, amino, methylamino, dimethylamino, and phenoxy;

or one pair selected from R^{a} and R^{b}, and R^{b} and R^{c}, taken together, is —O—(CH₂)—O— or —O—CH₂—CH₂—O—;

or a pharmaceutically acceptable salt, a C₁₋₆ alkyl ester or amide, or a C₂₋₆ alkenyl ester or amide thereof.

A second aspect of the present invention is a composition comprising an active compound as described herein in a pharmaceutically acceptable carrier.

A third aspect of the present invention is a method of treating rheumatoid arthritis in a subject in need thereof, comprising administering to the subject an active compound as described herein in a treatment effective amount, along with the use of an active compound as described herein for the manufacture of a medicament for treating rheumatoid arthritis in a subject in need thereof.

A fourth aspect of the present invention is a method of treating multiple sclerosis in a subject in need thereof, comprising administering to said subject. an active compound as described herein in a treatment effective amount, along with the use of an active compound as described herein for the manufacture of a medicament for treating multiple sclerosis in a subject in need thereof.

A fifth aspect of the invention is a method of treating an autoimmune disease in a subject in need thereof, comprising administering to the subject an active compound as described herein in a treatment effective amount, along with the use of an active compound as described herein for the manufacture of a medicament for treating an autoimmune disease in a subject in need thereof, wherein the autoimmune disease is selected from the group consisting of systemic lupus erythematosus, type 1 diabetes mellitus, psoriasis, and atherosclerosis.

Other aspects of the present invention are disclosed herein and discussed in greater detail below.

### DETAILED DESCRIPTION OF CERTAIN EMBODIMENTS OF THE INVENTION

### A. Definitions

“Enantiomerically pure” as used herein means a stereomerically pure compound, or composition of a compound, the compound having one chiral center.

“Stereomerically pure” as used herein means a compound or composition thereof that comprises one stereoisomer of a compound and is substantially free of other stercoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. See, e.g., U.S. Pat. No. 7,189,715.

“Stable”, as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

“Alkyl” or “alkyl group,” as used herein, means a straight-chain (i.e., unbranched), branched, or cyclic hydrocarbon chain that is completely saturated. In certain embodiments, alkyl groups contain 1-3 carbon atoms. In still other embodiments, alkyl groups contain 2-3 carbon atoms, and in yet other embodiments alkyl groups contain 1-2 carbon atoms. In certain embodiments, the term “alkyl” or “alkyl group” refers to a cycloalkyl group, also known as carbocycle. Exemplary C₁₋₃ alkyl groups include methyl, ethyl, propyl, isopropyl, and cyclopropyl.

“Alkenyl” or “alkenyl group,” as used herein, refers to a straight-chain (i.e., unbranched), branched, or cyclic hydrocarbon chain that has one or more double bonds. In certain embodiments, alkenyl groups contain 2-4 carbon atoms. In still other embodiments, alkenyl groups contain 3-4 carbon atoms, and in yet other embodiments alkenyl groups contain 2-3 carbon atoms. According to another aspect, the term alkenyl refers to a straight chain hydrocarbon having two double bonds, also referred to as “diene.” In other embodiments, the term “alkenyl” or “alkenyl group” refers to a cycloalkenyl group. Exemplary C₂₋₄ alkenyl groups include —CH═CH₂, —CH₂CH═CH₂ (also referred to as allyl), —CH═CHCH₃, —CH₂CH₂CH═CH₂, —CH₂CH═CHCH₃, —CH═CH₂CH₂CH₃, —CH═CH₂CH═CH₂, and cyclobutenyl.

“Alkoxy”, or “alkylthio”, as used herein, refers to an alkyl group, as previously defined, attached to the principal carbon chain through an oxygen (“alkoxy”) or sulfur (“alkylthio”) atom.

“Methylene”, “ethylene”, and “propylene” as used herein refer to the bivalent moieties —CH₂—, —CH₂CH₂—, and —CH₂CH₂CH₂—, respectively.

“Ethenylene”, “propenylene”, and “butenylene” as used herein refer to the bivalent moieties —CH═CH—, —CH═CHCH₂—, —CH₂CH═CH—, —CH═CHCH₂CH₂—, —CH₂CH═CH₂CH₂—, and —CH₂CH₂CH═CH—, where each ethenylene, propenylene, and butenylene group can be in the cis or trans configuration. In certain embodiments, an ethenylene, propenylene, or butenylene group can be in the trans configuration.

“Alkylidene” refers to a bivalent hydrocarbon group formed by mono or dialkyl substitution of methylene. In certain embodiments, an alkylidene group has 1-6 carbon atoms. In other embodiments, an alkylidene group has 2-6, 1-5, 2-4, or 1-3 carbon atoms. Such groups include propylidene (CH₃CH₂CH═), ethylidene (CH₃CH═), and isopropylidene (CH₃(CH₃)CH═), and the like.

“Alkenylidene” refers to a bivalent hydrocarbon group having one or more double bonds formed by mono or dialkenyl substitution of methylene. In certain embodiments, an alkenylidene group has 2-6 carbon atoms. In other embodiments, an alkenylidene group has 2-6, 2-5, 2-4, or 2-3 carbon atoms. According to one aspect, an alkenylidene has two double bonds. Exemplary alkenylidene groups include CH₃CH═C═, CH₂═CHCH═, CH₂═CHCH₂CH═, and CH₂═CHCH₂CH═CHCH═.

“C₁₋₆ alkyl ester or amide” refers to a C₁₋₆ alkyl ester or a C₁₋₆ alkyl amide where each C₁₋₆ alkyl group is as defined above. Such C₁₋₆ alkyl ester groups are of the formula (C₁₋₆ alkyl)OC(═O)— or (C₁₋₆ alkyl)C(═O)O—. Such C₁₋₆ alkyl amide groups are of the formula (C₁₋₆ alkyl)NHC(═O)— or (C₁₋₆ alkyl)C(═O)NH—.

“C₂₋₆ alkenyl ester or amide” refers to a C₂₋₆ alkenyl ester or a C₂₋₆ alkenyl amide where each C₂₋₆ alkenyl group is as defined above. Such C₂₋₆ alkenyl ester groups are of the formula (C₂₋₆ alkenyl)OC(═O)— or (C₂₋₆ alkenyl)C(═O)O—. Such C₂₋₆ alkenyl amide groups are of the formula (C₂₋₆ alkenyl)NHC(═O)— or (C₂₋₆ alkenyl)C(═O)NH—.

“Treatment,” “treat,” and “treating” refer to reversing, alleviating, delaying the onset of, inhibiting the progress of, or preventing a disease or disorder as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

“Patient” or “subject”, as used herein, means an animal subject, preferably a mammalian subject (e.g., dog, cat, horse, cow, sheep, goat, monkey, etc.), and particularly human subjects (including both male and female subjects, and including neonatal, infant, juvenile, adolescent, adult and geriatric subjects).

“Pharmaceutically acceptable carrier” as used herein refers to a nontoxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, cyclodextrins, sodium carboxymethylcellulose, polyacrylates, waxes, polyethylene-polyoxypropylene-block polymers, polyethylene glycol and wool fat.

Unless indicated otherwise, nomenclature used to describe chemical groups or moieties as used herein follow the convention where, reading the name from left to right, the point of attachment to the rest of the molecule is at the right-hand side of the name. For example, the group “(C₁₋₃ alkoxy)C₁₋₃ alkyl,” is attached to the rest of the molecule at the alkyl end. Further examples include methoxyethyl, where the point of attachment is at the ethyl end, and methylamino, where the point of attachment is at the amine end.

Unless indicated otherwise, where a bivalent group is described by its chemical formula, including two terminal bond moieties indicated by “—,” it will be understood that the attachment is read from left to right.

Unless otherwise stated, structures depicted herein are also meant to include all enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C— or ¹⁴C-enriched carbon are within the scope of this invention. Such compounds are useful, for example, as analytical tools or probes in biological assays.

### B. Active Compounds

As described herein, active compounds of the invention may optionally be substituted with one or more substituents, such as are illustrated generally above, or as exemplified by particular classes, subclasses, and species of the invention. In general, the term “substituted” refers to the replacement of hydrogen radicals in a given structure with the radical of a specified substituent. Unless otherwise indicated, a substituted group may have a substituent at each substitutable position of the group, and when more than one position in any given structure may be substituted with more than one substituent selected from a specified group, the substituent may be either the same or different at every position.

As noted above, the present invention provides enantiomerically pure compounds, or active compounds, of Formula I:

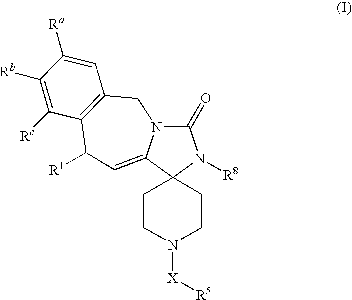

or more particularly Formula Ia or Formula Ib:

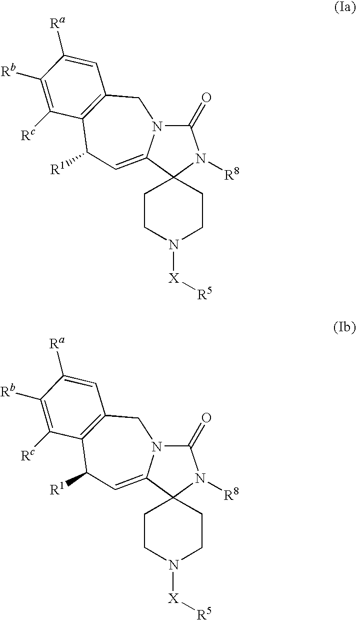

wherein:
R¹ is C₁₋₃ alkyl;
X is methylene, ethylene, propylene, ethenylene, propenylene, or butenylene;
R⁵ is phenyl, pyrrolyl, benzimidazolyl, oxazolyl, isoxazolyl, imidazothiazolyl, quinolinyl, isoquinolinyl, indazolyl, pyridinyl, imidazopyridinyl, indolyl, benzotriazolyl, imidazolyl, benzofuranyl, benzothiadiazolyl, pyridimidinyl, benzopyranonyl, thiazolyl, thiadiazolyl, furyl, thienyl, pyrazolyl, quinoxalinyl, or naphthyl, and substituted with between 0 and 5 substituents independently selected from C₁₋₄ alkyl, C₁₋₃ alkoxy, hydroxyl, C₁₋₃ alkylthio, cyclopropyl, cyclopropylmethyl, trifluoromethoxy, 5-methylisoxazolyl, pyrazolyl, benzyloxy, acetyl, (cyanyl)C₁₋₃ alkyl, (phenyl)C₂₋₃ alkenyl; and halo;
R⁸ is H, methyl, ethyl, propyl, (C₁₋₃ alkoxy)C₁₋₃ alkyl, (C₁₋₃ alkylthio)C₁₋₃ alkyl, C₁₋₃ hydroxyalkyl, phenyl, benzyl, furyl, pyrrolyl, imidazolyl, pyrazolyl, pyrrolyl, isothiazolyl, isooxazolyl, pyridyl, and thienyl;
wherein R⁸ is substituted with between 0 and 3 substituents independently selected from methyl, ethyl, halo, hydroxyl, C₁₋₃ alkoxy, C₁₋₃ alkylthio, (C₁₋₃ alkoxy)C₁₋₃ alkyl, (C₁₋₃ alkylthio)C₁₋₃ alkyl, C₁₋₃ hydroxyalkyl, (C₁₋₃ mercaptoalkyl)phenyl, benzyl, furyl, imidazolyl, pyrazolyl, pyrrolyl, isothiazolyl, isooxazolyl, pyridyl, and thienyl; and
each of R^{a}, R^{b}, and R^{c} is independently selected from hydrogen, hydroxyl, methoxy, benzyloxy, fluoro, chloro, amino, methylamino, dimethylamino, and phenoxy;
or one pair selected from R^{a} and R^{b}, and R^{b} and R^{c}, taken together, is —O—(CH2)-O— or —O—CH₂—CH₂—O—;
   or a pharmaceutically acceptable salt, a C₁₋₆ alkyl ester or amide, or a C₂₋₆ alkenyl ester or amide thereof.

In some embodiments of the foregoing:
R¹ is C₁₋₂ alkyl;
R⁵ is phenyl, pyrrolyl, benzimidazolyl, oxazolyl, isoxazolyl, imidazothiazolyl, quinolinyl, isoquinolinyl, indazolyl, pyridinyl, imidazopyridinyl, indolyl, benzotriazolyl, imidazolyl, benzofuranyl, benzothiadiazolyl, pyridimidinyl, benzopyranonyl, thiazolyl, thiadiazolyl, furyl, thienyl, pyrazolyl, quinoxalinyl, or naphthyl, and substituted with between 0 and 5 substituents independently selected from C₁₋₄ alkyl, C₁₋₃ alkoxy, hydroxyl, C₁₋₃ alkylthio, cyclopropyl, cyclopropylmethyl, trifluoromethoxy, 5-methylisoxazolyl, pyrazolyl, benzyloxy, acetyl, (cyanyl)C₁₋₃ alkyl, (phenyl)C₂₋₃ alkenyl; and halo;
R⁸ is, methyl, ethyl, or propyl, wherein R⁸ is substituted with from 0 and 3 hydroxyl substituents;
X is methylene or ethylene; and
R^{a} R^{b} and R^{c} are each independently selected from the group consisting of H and methoxy.
   or a pharmaceutically acceptable salt, a C₁₋₆ alkyl ester or amide, or a C₂₋₆ alkenyl ester or amide thereof.

In some embodiments of the foregoing:
R¹ is methyl;
R⁵ is phenyl, pyrrolyl or pyrazolyl, each of which is substituted 0, 1 or 2 times with methyl;
R⁸ is ethyl;
X is methylene;
R^{a} and R^{c} are each methoxy; and
R^{b} is H;
   or pharmaceutically acceptable salt thereof.

In particular embodiments of the foregoing, the compound is:

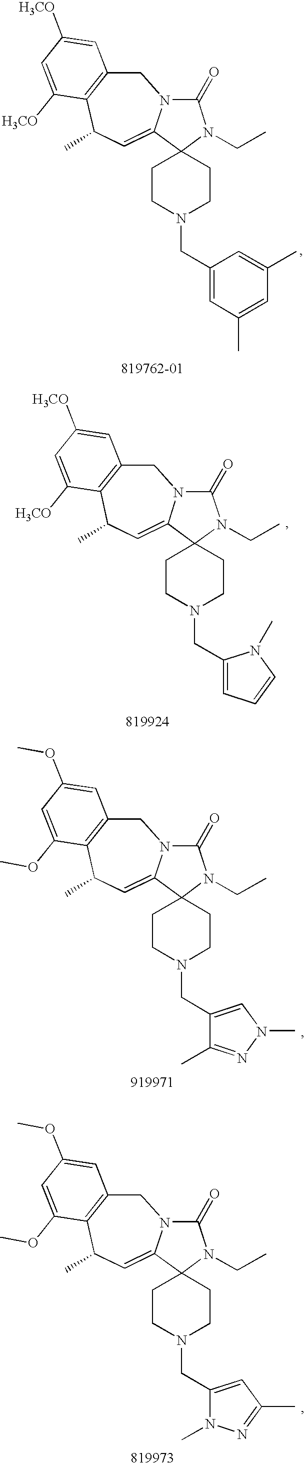

or a pharmaceutically acceptable salt thereof.

Active compounds of the present invention include pharmaceutically acceptable salts of the foregoing. Pharmaceutically acceptable salts include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., magnesium), ammonium and N+(C₁₋₄ alkyl)₄ salts. This invention also envisions the quaternization of any basic nitrogen-containing groups of the compounds disclosed herein. Water- or oil-soluble or dispersible products may be obtained by such quaternization.

### C. Pharmaceutical Formulations

Active compounds of the present invention can be combined with a pharmaceutically acceptable carrier to provide pharmaceutical formulations thereof. The particular choice of carrier and formulation will depend upon the particular route of administration for which the composition is intended.

The compositions of the present invention may be suitable for oral, parenteral, inhalation spray, topical, rectal, nasal, buccal, vaginal or implanted reservoir administration, etc. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a nontoxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically transdermal patches may also be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

For ophthalmic use, the pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, the pharmaceutically acceptable compositions of this invention are formulated for oral administration.

### D. Subjects and Methods of Use

Active compounds of the present invention may be administered to patients or subjects to treat a variety of different condition, particularly patients or subjects afflicted with:
(a) rheumatoid arthritis;
(b) multiple sclerosis;
(c) systemic lupus erythematosus (see, e.g., T-bet regulates IgG class switching and pathogenic auto Ab production, Proc. Natl. Acad. Sci. USA 99(8): 5545-50 (2002); Imbalance of Th1/Th2 transcription factors in patients with lupus nephritis, Rheumatology (Oxford) 45(8): 951-7 (2006));
(d) type 1 diabetes (see, e.g., Identification of a novel type 1 diabetes susceptibility gene, T-bet, Human Genetics 111(3): 177-84 (2004); T-bet controls autoaggressive CD8 lymphocyte response in type I diabetes, J. Exp. Med. 199(8): 1153-62 (2004));
(e) psoriasis (see, e.g., J. Mol. Med. 81(8): 471-80 (2003)); and
(f) atherosclerosis (see, e.g., Proc. Natl. Acad. Sci. USA 102(5): 1596-601 (2005)).

Active compounds may be administered to subjects by any suitable route, including orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

The active compounds are administered to the subjects in a treatment effective, or therapeutically effective, amount. The amount of the compounds of the present invention that may be combined with the carrier materials to produce a composition in a single dosage form will vary depending upon the host treated, and the particular route of administration. Preferably, the compositions should be formulated so that a dosage of between 0.01-100 mg/kg body weight/day of the inhibitor can be administered to a patient receiving these compositions. In certain embodiments, the compositions of the present invention provide a dosage of between 0.01 mg and 50 mg is provided. In other embodiments, a dosage of between 0.1 and 25 mg or between 5 mg and 40 mg is provided.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

In order that the invention described herein may be more fully understood, the following examples are set forth. It should be understood that these examples are for illustrative purposes only and are not to be construed as limiting this invention in any manner.

### Examples 1-41

### Synthesis of Compounds

Microwave assisted reactions were carried out using an Emrys Liberator instrument supplied by Biotage Corporation. Solvent removal was carried out using either a Büchi rotary evaporator or a Genevac centrifugal evaporator. Analytical and preparative chromatography was carried out using a Waters autopurification instrument using either normal phase or reverse phase HPLC columns, under either acidic, neutral, or basic conditions. Compounds were estimated to be >90% pure, as determined by area percent of ELSD chromatograms. NMR spectra were recorded using a Varian 300 MHz spectrometer.

General methods and experimentals for preparing compounds of the present invention are set forth below. In certain cases, a particular compound is described by way of example. However, it will be appreciated that in each case a series of compounds of the present invention were prepared in accordance with the schemes and experimentals described below.

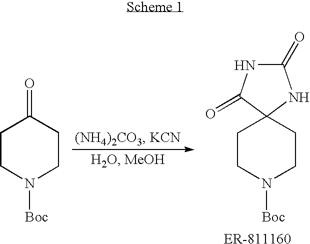

ER-811160. As depicted in Scheme 1 above, a solution of potassium cyanide (22.5 g, 0.335 mol) in water (50 mL) was added dropwise over 5 minutes to a solution of 1-Boc-piperidone (32.48 g, 0.1598 mol) and ammonium carbonate (33.8 g, 0.351 mol) in water (90 mL) and methanol (110 mL). An off-white precipitate began to form soon after addition was complete. The reaction flask was sealed and the suspension stirred at room temperature for 72 hours. The resultant pale yellow precipitate was filtered and was washed with small portions of water to give ER-811160 (37.1 g, 86%) as a colorless solid.

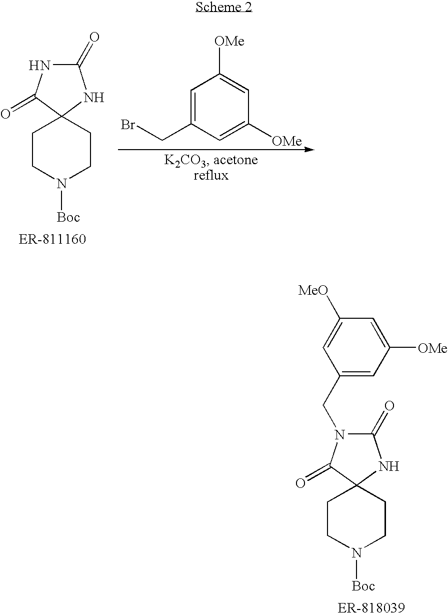

ER-818039. As depicted in Scheme 2 above, a suspension of ER-811160 (30.0 g, 0.111 mol), 3,5-dimethoxybenzyl bromide (30.9 g, 0.134 mol), and potassium carbonate (18.5 g, 0.134 mol) in acetone (555 mL) was heated under reflux overnight. The reaction solution was cooled to room temperature, filtered and concentrated in vacuo. The crude orange residue was dissolved in a minimal amount of MTBE (250 mL). A small amount of hexanes was added (50 mL) and the product allowed to precipitate out (over ˜2 hours) as a colorless solid which was isolated by vacuum filtration. The filter cake was washed with small amounts of MTBE, and dried in vacuo to provide ER-818039 (39.6 g, 85%) as a colorless solid.

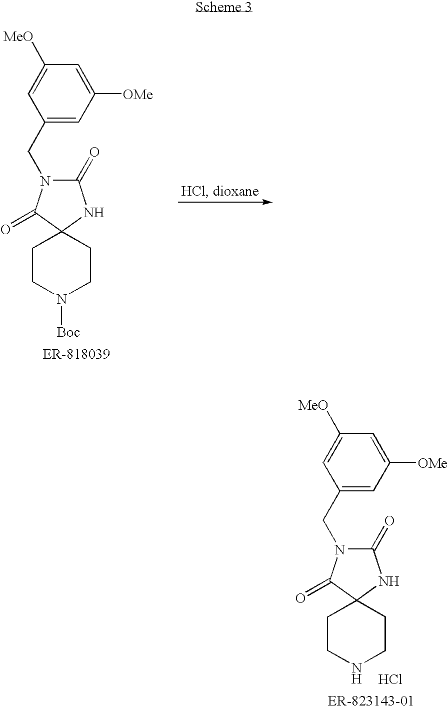

ER-823143-01. As depicted in Scheme 3 above, to a 1-neck round-bottom flask containing ER-818039 (2.15 g, 0.00512 mol) was slowly added a solution of 4N HCl in 1,4-dioxane (3.8 mL, 0.049 mol). The starting material slowly dissolved over 20 minutes and a colorless precipitate formed after 30 minutes. MTBE (3 ml) was then added. After 2 hours, the reaction was filtered and washed with MTBE, which provided ER-823143-01 (1.81 g, 99%) as a colorless solid.

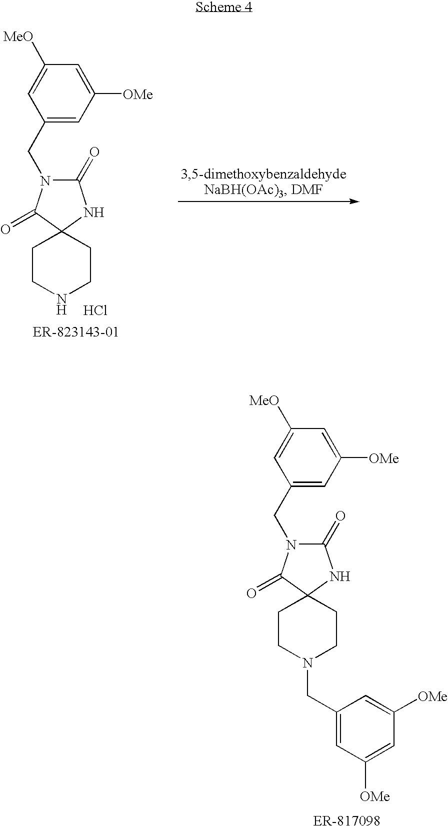

ER-817098: As depicted in Scheme 4 above, to a suspension of ER-823143-01 (41.5 mg, 0.000117 mol) and 4 Å molecular sieves in 1,2-dimethoxyethane (0.5 mL, 0.004 mol) under an atmosphere of nitrogen was added 3,5-dimethoxybenzaldehyde (21.3 mg, 0.000128 mol) followed by triethylamine (16.2 μL, 0.000117 mol). The reaction was stirred for 1 hour. Sodium triacetoxyborohydride (34.6 mg, 0.000163 mol) was added, and the reaction was stirred overnight. Silica gel flash chromatography yielded ER-817098 (45.3 mg, 83%) as a colorless solid.

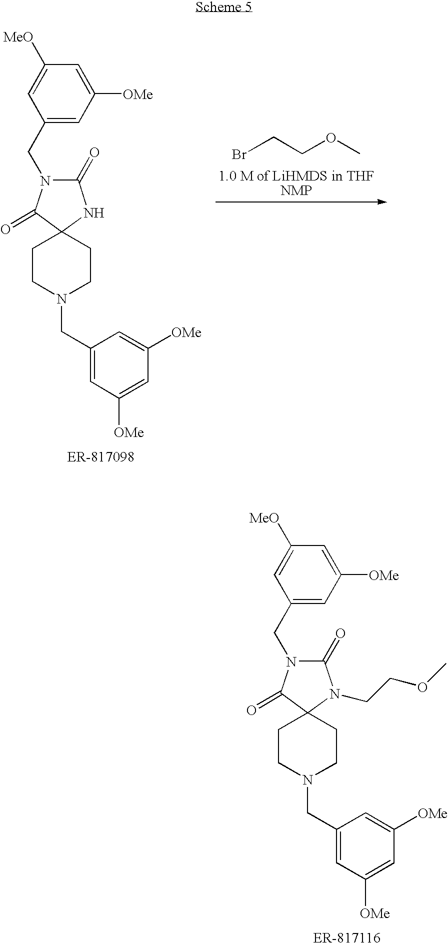

ER-817116: As depicted in Scheme 5 above, to a solution of ER-817098-00 (50.0 mg, 0.000106 mol) and 1-bromo-2-methoxyethane (15.6 μL, 0.000160 mol) in N-methylpyrrolidinone (1.0 mL, 0.010 mol) was added 1.0 M lithium hexamethyldisilazide solution in tetrahydrofuran (0.16 mL). The temperature was increased to at 80° C. and the reaction mixture stirred overnight. The reaction mixture was cooled to room temperature, quenched with water and then extracted several times with MTBE. The MTBE extracts were combined and washed with water (2×) and brine (1×). The organic layer was dried over magnesium sulfate, filtered, and concentrated in vacuo. Flash chromatography provided ER-817116 (32.2 mg, 58%) as colorless oil.

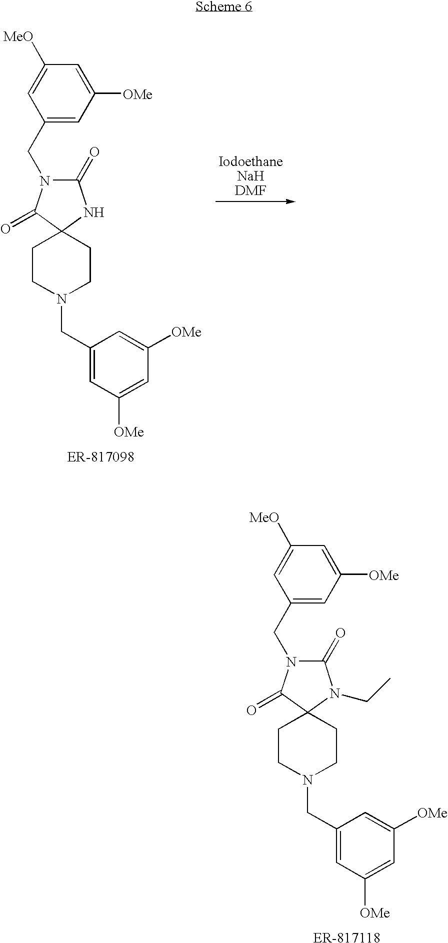

ER-817118: As depicted in Scheme 6 above, to a solution of ER-817098 (2.85 g, 0.00607 mol) in N,N-dimethylformamide (15 mL) was added sodium hydride (364 mg, 0.00910 mol) followed by iodoethane (758 μL, 0.00910 mol). The reaction mixture was stirred overnight. Water was very slowly added and the reaction mixture was extracted several times with MTBE. The MTBE extracts were combined and washed with water (2×) and brine (1×). The organic layer was dried over magnesium sulfate, filtered, and concentrated in vacuo. Flash chromatography using ethyl acetate as eluent provided ER-817118 (2.89 g, 96%) as a colorless oil.

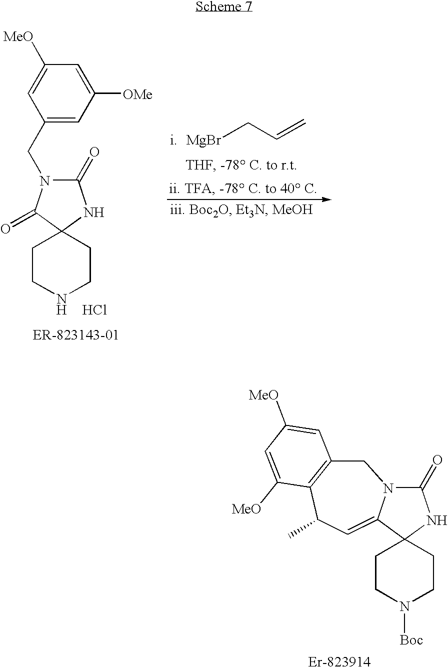

ER-823914: As depicted in Scheme 7 above, to a solution of ER-823143-01 (5.03 g, 0.0141 mol) in tetrahydrofuran (30.0 mL, 0.370 mol) at −78° C. was slowly added 1.0 M of allylmagnesium bromide in ether (71 mL). The reaction mixture was warmed to room temperature and stirred overnight. The reaction mixture was cooled to −78° C., treated dropwise with trifluoroacetic acid (21.8 mL, 0.283 mol), and then concentrated in vacuo to a small residual volume. Triethylamine was added to neutralize residual TFA and the mixture then concentrated in vacuo to dryness. The residual red oil was dissolved in methanol (138 mL, 3.41 mol) and treated with di-tert-butyldicarbonate (3.34 g, 0.0148 mol) followed by triethylamine (2.38 mL, 0.0169 mol) and stirred overnight at room temperature. The reaction mixture was concentrated in vacuo and purified by flash chromatography (eluent: 50% hexanes in ethyl acetate) to provide ER-823914 (3.25 g, 52%) as a colorless solid.

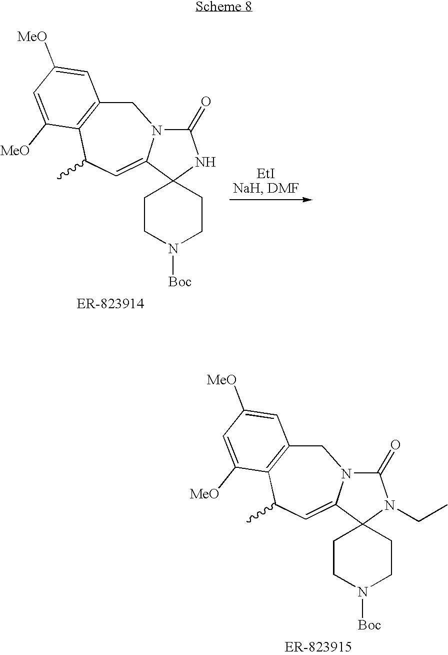

ER-823915: As depicted in Scheme 8 above, to a solution of ER-823914 (2.20 g, 0.00496 mol) in N,N-Dimethylformamide (12.4 mL, 0.160 mol) was added sodium hydride (298 mg, 0.00744 mol) followed by iodoethane (607 μL, 0.00744 mol). The reaction mixture was stirred overnight then quenched with water and extracted several times with MTBE. The MTBE extracts were combined and washed with water and brine. The organic layer was dried over magnesium sulfate, filtered, and concentrated in vacuo. Flash chromatography (eluent: 40% hexanes in ethyl acetate) provided ER-823915 (0.80 g, 34%) as a colorless foam.

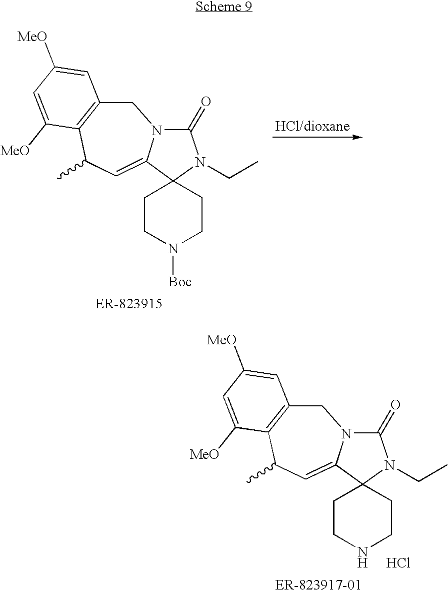

ER-823917-01: As depicted in Scheme 9 above, ER-823915 (799.2 mg, 0.001695 mol) was dissolved in a solution of 4 M hydrogen chloride in 1,4-dioxane (10 mL). The reaction mixture was stirred overnight and then concentrated in vacuo to provide ER-823917-01 (0.69 g, quantitative) as an orange solid.

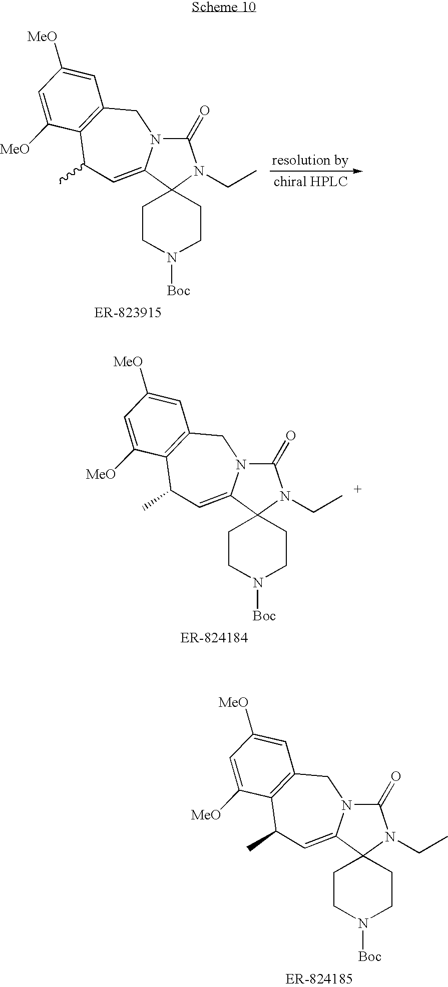

ER-824184 & ER-824185: As depicted in Scheme 10 above, a solution of ER-823915 (200 mg) in acetonitrile (1 ml) was injected onto a CHIRALPAK® AS-H SFC column (30 mm×250 mm, 5 micron particle size) and eluted with 95:5 n-heptane: i-propanol at a flow rate of 40 ml/min. Eluted fractions were detected using a UV detector with the wavelength set at 290 m-n. The first eluting fraction was isolated and concentrated by rotary evaporation in vacuo to afford ER-824184; the second eluting fraction was isolated and concentrated by rotary evaporation in vacuo to afford ER-824185.

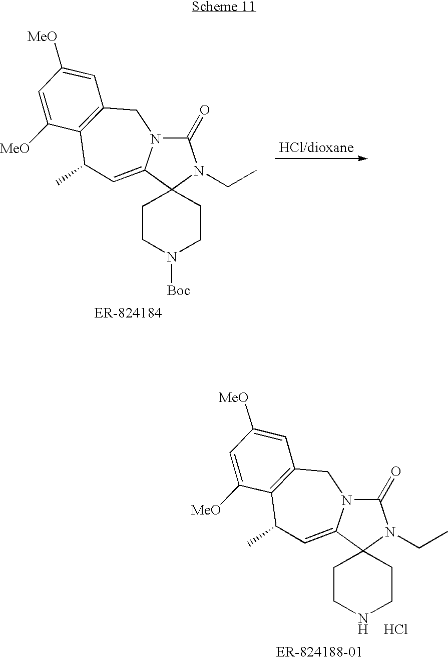

ER-824188-01: As depicted in Scheme 11 above, ER-824184 (25.33 g, 0.05371 mol) was dissolved in a solution of 4 M hydrogen chloride in 1,4-dioxane (135 mL). The reaction mixture was stirred overnight and then concentrated in vacuo to provide ER-824188-01 (21.9 g, quantitative) as an orange solid. Single crystal X-ray diffraction analysis of ER-824188-01 showed the absolute configuration of the stereocenter to be S, as depicted in Scheme 11.

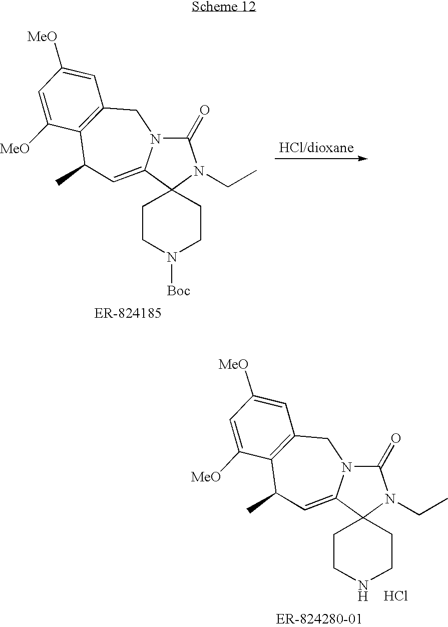

ER-824280-01: As depicted in Scheme 12 above, ER-824185 (457.2 mg, 0.0009695 mol) was dissolved in a solution of 4 M hydrogen chloride in 1,4-dioxane (2.5 mL). The reaction mixture was stirred overnight and then concentrated in vacuo to provide ER-824280-01 (383.2 mg, 97%) as an orange solid. Single crystal X-ray diffraction analysis of a Mosher amide derivative of ER-824188-01 showed the absolute configuration of the stereocenter to be R, as depicted in Scheme 11.

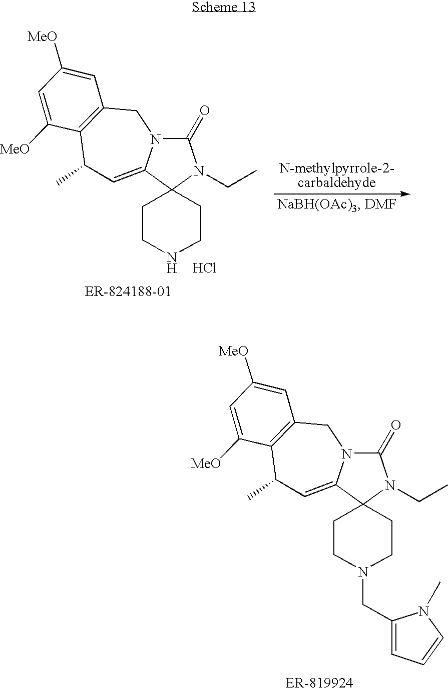

ER-819924: As depicted in Scheme 13 above, ER-824188-01 (62.4 mg, 0.000153 mol) and N-methylpyrrole-2-carbaldehyde (0.000229 mol) were dissolved/suspended in N,N-dimethylformamide (0.62 mL). After stirring for 30 minutes, sodium triacetoxyborohydride mg, 0.000214 mol) was added. The reaction mixture was stirred overnight then purified by reverse phase chromatography to afford ER-819924 (71.1 mg, 83.4%) as an oil.

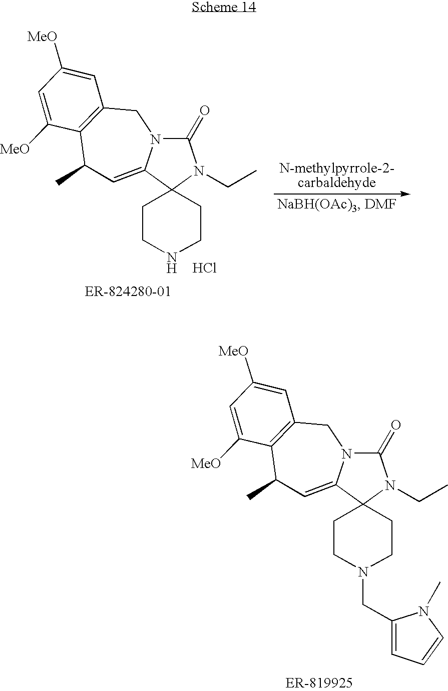

ER-819925: As depicted in Scheme 14 above, ER-824280-01 (59.5 mg, 0.000146 mol and N-methylpyrrole-2-carbaldehyde (0.000219 mol) were dissolved/suspended in N,N′-dimethylformamide (0.60 mL). After stirring for 30 minutes, sodium triacetoxyborohydride (45.6 mg, 0.000204 mol) was added. The reaction mixture was stirred overnight then purified by reverse phase chromatography to afford ER-819925 (51.9 mg, 76.6%) as an oil.

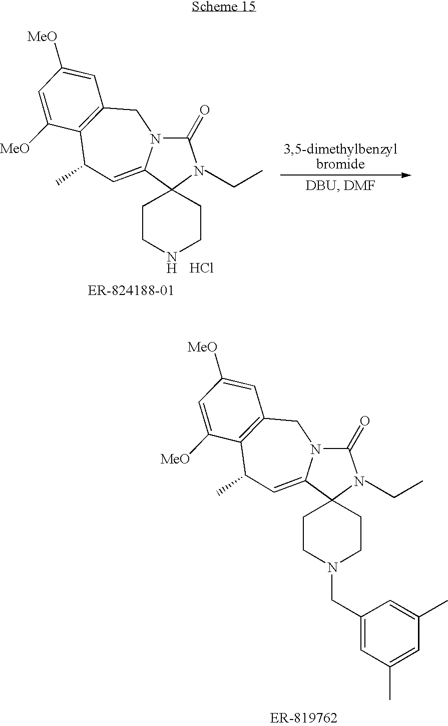

ER-819762: As depicted in Scheme 15 above, a solution of ER-824188-01 (5.7 g, 0.0140 mol), 1,8-diazabicyclo[5.4.0]undec-7-ene (4.4 mL, 0.029 mol) and 3,5-dimethylbenzyl bromide (4.7 g, 0.024 mol) in N,N-dimethylformamide (50 mL) was heated at 97 C overnight. An aqueous work-up and purification by flash chromatography provided ER-819762 (4.86 g, 71%) as colorless solid.

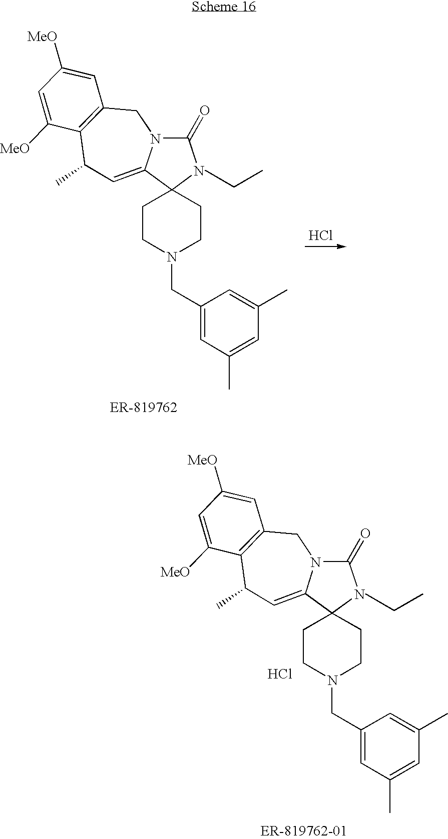

ER-819762-01: As depicted in Scheme 16 above, a solution of ER-819762 (4.77 g, 0.00974 mol), Acetonitrile (10 mL) and 1M HCl in Water (11 mL) was stirred at room temperature for approximately 5 minutes. The solution was concentrated to provide ER-819762-01 (5.1 g, quantitative) as a colorless crystalline solid after lyophilization. Single crystal X-ray diffraction analysis of ER-819762-01 showed the absolute configuration of the stereocenter to be S, as depicted in Scheme 16.

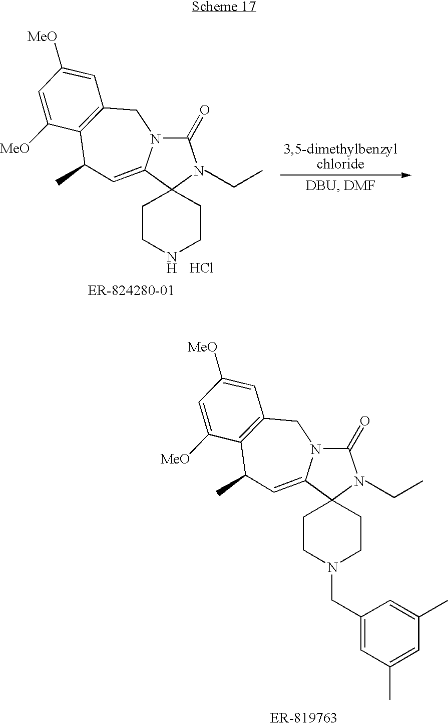

ER-819763: As depicted in Scheme 17 above, a solution of ER-824280-01 (66.9 g, 0.1640 mol), 1,8-diazabicyclo[5.4.0]undec-7-ene (54 mL, 0.361 mol) and 3,5-dimethylbenzyl chloride (42.4 g, 0.213 mol) in N-Methylpyrrolidinone (669 mL) was heated at 72 C for 2 hours. After cooling, water was added to precipitate the desired product. Filtration and drying under vacuum provided ER-819763 (74.4 g, 92%) as colorless solid.

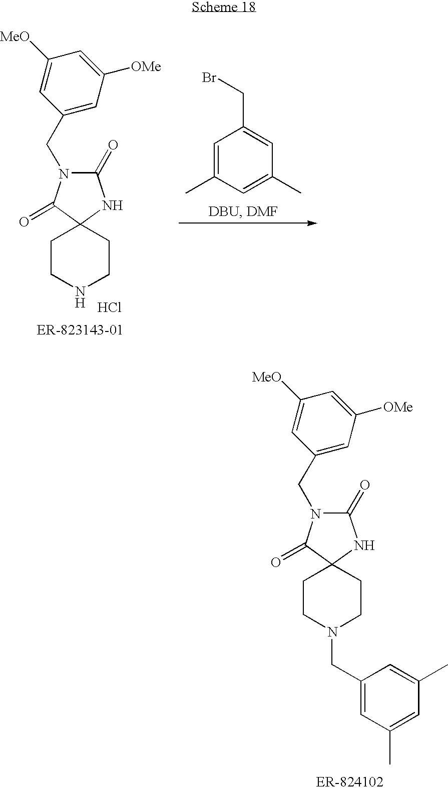

ER-824102: As depicted in Scheme 18 above, to a solution of ER-823143-01 (4.00 g, 0.0112 mol) in N,N-dimethylformamide (25 mL) at room temperature was added alpha-bromomesitylene (3.13 g, 0.0157 mol) followed by DBU (4.37 mL, 0.0292 mol). After stirring for 1 hour, reaction was quenched with half-saturated aq. NH₄Cl, diluted with ethyl acetate, and stirred for 1 h to give two clear layers. Organic layer was separated, aq. layer was extracted with ethyl acetate (2×). Combined extracts were dried over Na₂SO₄, filtered, and concentrated in vacuo. Crystallization from MTBE afforded ER-824102 (4.30 g, 87%) as a colorless solid.

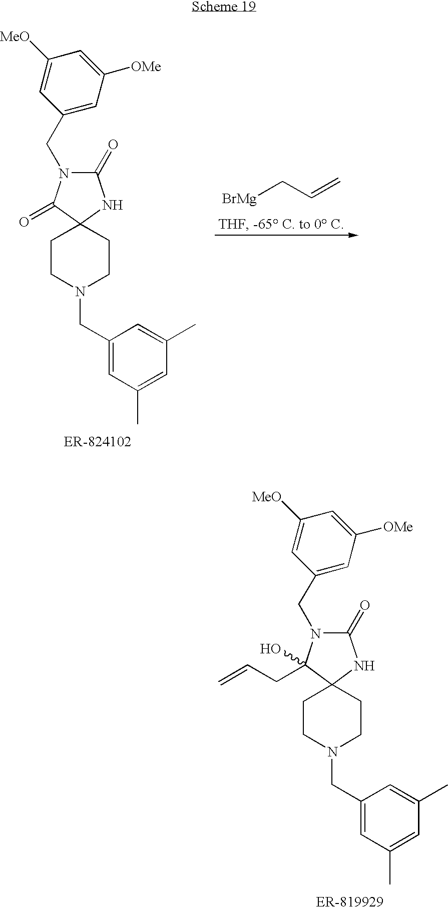

ER-819929: As depicted in Scheme 19 above, to a solution of ER-824102 (3.72 g, 0.0085 mol) in tetrahydrofuran (35 mL) at −65° C. was added 1.0 M allylmagnesium bromide in ether (25.5 mL, 0.0255 mol) over 10 min keeping internal temperature below −50° C. The reaction mixture was allowed to warm to 0° C. After 3 h at 0° C., reaction was quenched with saturated aq. NH₄Cl, diluted with ethyl acetate and water, stirred for 10 min to give two clear layers. Organic layer was separated, aq. layer was extracted with ethyl acetate. Combined extracts were washed with water, brine, dried over Na₂SO₄, filtered, concentrated in vacuo to give crude product ER-819929 (4.15 g, quantitative) as a colorless solid that was used for next step without further purification.

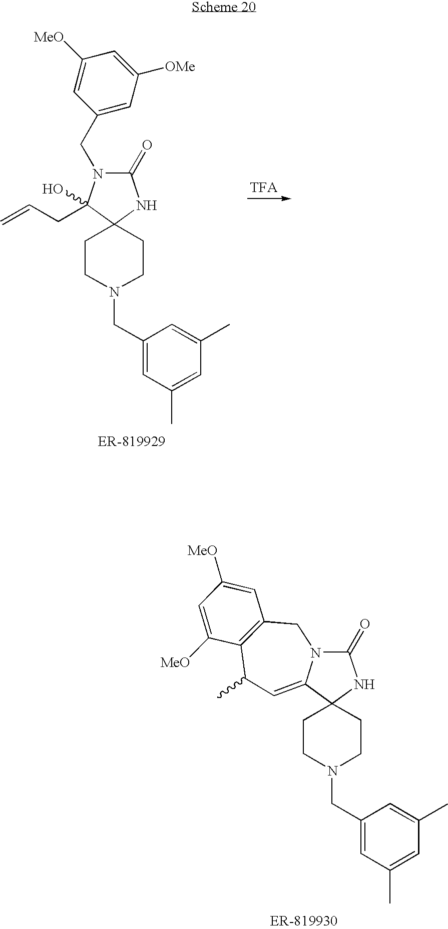

ER-819930: As depicted in Scheme 20 above, a solution of ER-819929 (37 mg, 0.000077 mol) in trifluoroacetic acid (0.5 mL) was stirred at room temperature for 16 hours. Dark brown-red reaction mixture was diluted with EtOAc (5 mL), neutralized with sat aq NaHCO₃ (5 mL, careful: gas evolution). Two-layer mixture was stirred for 10 min to give two clear, almost colorless layers. The organic layer was separated; the aq layer was extracted with EtOAc. Combined organic extracts were dried over Na₂SO₄, filtered, concentrated in vacuo. Purification by flash chromatography eluting with 1:1 Heptane-EtOAc, 1:3 Heptane-EtOAc, 100% EtOAc afforded ER-819930 (26 mg, 73%) as a colorless solid.

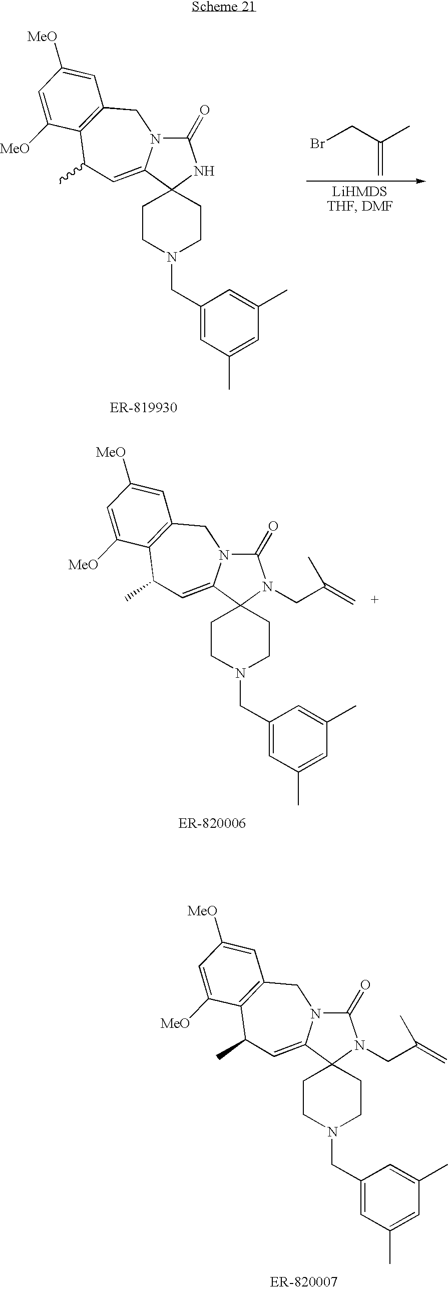

ER-820006 and ER-820007: As depicted in Scheme 21 above, to a solution of ER-819930 (110 mg, 0.000238 mol) and methallyl bromide (72 μL, 0.000715 mol) in DMF (1.5 mL,) was added 1.0 M lithium hexamethyldisilazide solution in tetrahydrofuran (0.52 mL, 0.00052 mol). After stirring for 18 h at rt, reaction mixture was diluted with MTBE, quenched with half-saturated aq NH₄Cl. Aq. layer was separated, extracted with MTBE. Combined extracts were dried over Na₂SO₄, filtered, concentrated in vacuo. Purification by flash chromatography eluting with 3:2 Heptane-EtOAc, 1:1 Heptane-EtOAc furnished racemic product (68 mg, 55%) as a colorless oil. Racemic product (55 mg) was subjected to chiral HPLC on Chiralpak AS column eluting with heptane-isopropanol (9:1) to afford first eluting enantiomer ER-820006 (21 mg, 38%, [α]_{D}=+83.7° (c=0.35, CHCl₃) and second eluting enantiomer ER-820007 (23 mg, 42%, [α]_{D}=−74.2° (c=0.38, CHCl₃). Absolute stereochemistry was assigned tentatively based on analogy in optical rotation and chiral HPLC retention time with ER-819762/ER-819763 pair of enantiomers.

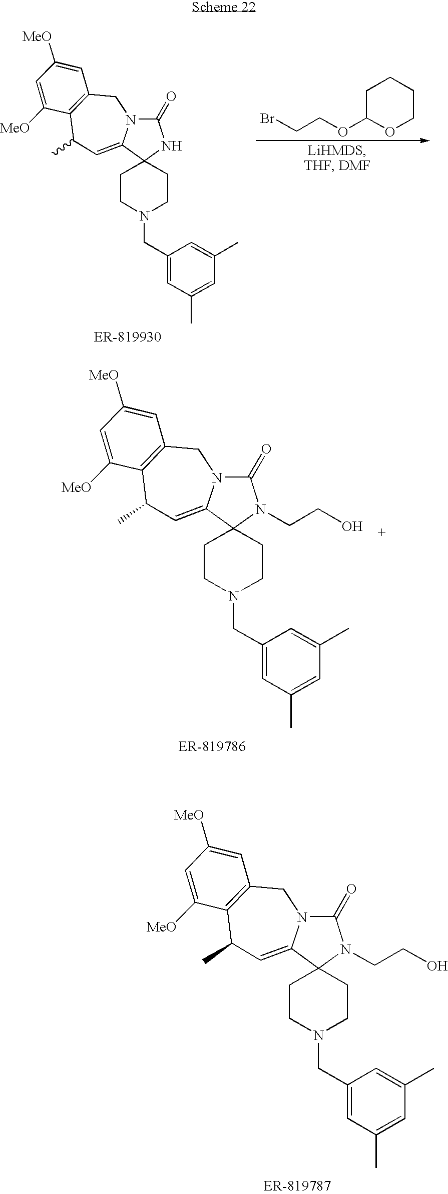

ER-819786 and ER-819787: As depicted in Scheme 22 above, a 5 mL microwave reactor vial equipped with a stir bar was charged with ER-819930 (110 mg, 0.000238 mol), DMF (1.5 mL), 2-(2-bromoethoxy)tetrahydro-2H-pyran (108 μL, 0.000715 mol) and 1.00 M of lithium hexamethyldisilazide in tetrahydrofuran (520 μL, 0.00052 mol). The reactor vial was microwaved at 200° C. for 15 min. More 2-(2-bromoethoxy)tetrahydro-2H-pyran (108 μL, 0.000715 mol) and 1.00 M of lithium hexamethyldisilazide in tetrahydrofuran (520 μL, 0.00052 mol) were added, and reaction mixture was heated by microwave irradiation at 200° C. for another 15 min. Purification by preparative reverse phase HPLC provided racemic product (25 mg, 21%) as a colorless glassy oil. Racemic product (17 mg) was subjected to chiral HPLC on Chiralpak AS column eluting with heptane-isopropanol (9:1) to afford first eluting enantiomer ER-819786 (7.2 mg, 42%, [α]_{D}=+72.0° (c=0.1, CHCl₃) and second eluting enantiomer ER-819787 (7.5 mg, 44%, [α]_{D}=−73.0° (c=0.1, CHCl₃). Absolute stereochemistry was assigned tentatively based on analogy in optical rotation and chiral HPLC retention time with ER-819762/ER-819763 pair of enantiomers.

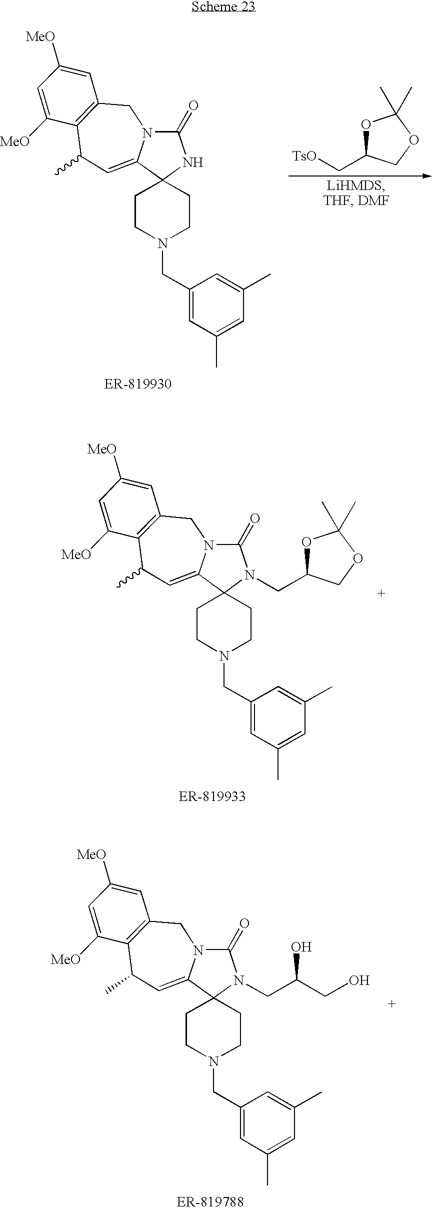

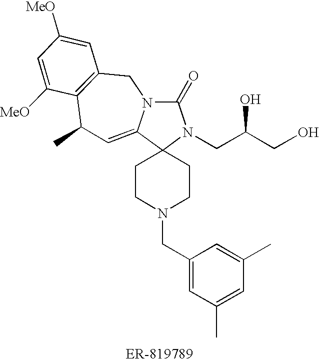

ER-819993 and ER-819994: As depicted in Scheme 23 above, a 5 mL microwave reactor vial equipped with a stir bar was charged with ER-819930 (110 mg, 0.000238 mol), DMF (1.5 mL), ((4S)-2,2-dimethyl-1,3-dioxolan-4-yl)methyl 4-methylbenzenesulfonate (205 mg, 0.000715 mol) and 1.00 M of lithium hexamethyldisilazide in tetrahydrofuran (520 μL, 0.00052 mol). The reactor vial was heated by microwave irradiation at 200° C. for 15 min. More ((4S)-2,2-dimethyl-1,3-dioxolan-4-yl)methyl 4-methylbenzenesulfonate (157 mg, 0.000548 mol) and 1.00 M of lithium hexamethyldisilazide in tetrahydrofuran (477 μL, 0.000477 mol) were added, and reaction mixture was heated by microwave irradiation at 200° C. for another 15 min. Purification by preparative reverse phase HPLC provided acetonide ER-819993 (40 mg, 30%) and diol material (18 mg, 14%) as 1:1 mixtures of diastereomers. Separation of diastereomeric diols by chiral HPLC on Chiralpak AS column eluting with heptane-isopropanol (9:1) afforded the first eluting diastereomer ER-819788 (5.0 mg) and the second eluting diastereomer ER-819789 (5.2 mg). Absolute stereochemistry was assigned tentatively based on analogy in chiral HPLC retention time with ER-819762/ER-819763 pair of enantiomers.

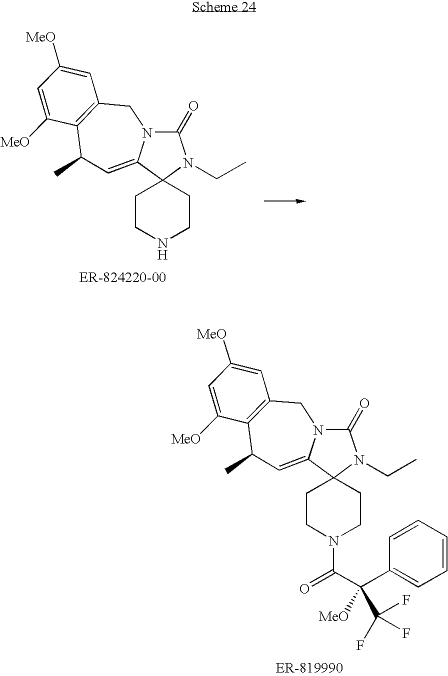

ER-81990: As depicted in Scheme 24 above, a solution of ER-824220-00 (51.8 mg, 0.000139 mol), triethylamine (97 μL, 0.00070 mol), 4-dimethylaminopyridine (3.4 mg, 0.000028 mol) and (R)-(−)-α-Methoxy-α-trifluoromethylphenylacetyl chloride (0.052 mL, 0.00028 mol) in Methylene Chloride (500 μL) was stirred at room temperature for 5 hours. Purification by flash chromatography, followed by crystallization from ethyl acetate/heptane/pentane provided ER-819990 (49.2 mg, 60%) as crystals.

Compounds that are exemplified in subsequent sections, and in Tables 1-2 below, but not depicted explicitly above, can be synthesized using general methods consistent with Scheme 13 and/or Scheme 15. For compounds exemplified in hydrochloride salt form, these can be prepared by subjecting the corresponding free base to the general conditions described in Scheme 16.

TABLE 1
Analytical Data for Exemplary Compounds of Formula I

| Example # | Structure | ER-# | Analytical Data |
|---|---|---|---|
| 1 | 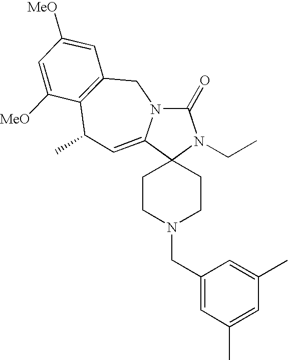 | 819762-01 HCl Salt | NMR ¹H (400 MHz, CD₃OD) δ 7.18 (s, 2 H), 7.15 (s, 1 H), 6.49 (d, J = 2.6 Hz, 1 H), 6.42 (d, J = 2.3 Hz, 1 H), 5.18 (br s, 1 H), 4.70 (d, J = 14.4 Hz, 1 H), 4.62 (d, J = 14.4 Hz, 1 H), 4.32 (s, 2 H), 4.27-4.19 (m, 1 H), 3.80 (s, 3 H), 3.78 (s, 3 H), 3.50-3.38 (m, 4 H), 3.26 (br s, 2 H), 2.36 (s, 6 H), 2.31-2.17 (m, 2 H), 1.97 (br d, J = 14.4 Hz, 1 H), 1.79 (br d, J = 14.4 Hz, 1 H), 1.38 (d, J = 7.3 Hz, 3 H), 1.16 (t, J = 7.0 Hz, 3 H) M/Z (ES+) Calc.: 489.30 Found: 490.40 (M + H) Analytical HPLC: Method A1 SunFire MS C18 (4.6 × 100 mm) 5 um Retention Time: 11.21 min Chiral HPLC Method C1 CHIRALPAK IA (0.46 × 25 cm) Retention Time: 6.819 min |
| 2 | 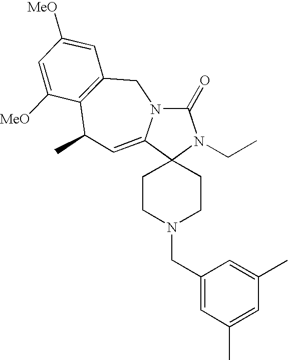 | 819763-00 Salt Free | NMR ¹H (400 MHz, CDCl₃) δ 6.94 (s, 2 H), 6.90 (s, 1 H), 6.44 (d, J = 2.6 Hz, 1 H), 6.41 (d, J = 2.3 Hz, 1 H), 5.02 (d, J = 8.5 Hz, 1 H), 4.81 (d, J = 14.1 Hz, 1H), 4.58 (d, J = 14.4 Hz, 1 H) 4.17-4.09 (m, 1 H), 3.79 (s, 3 H), 3.78 (s, 3 H), 3.49 (s, 2 H), 3.51-3.26 (m, 1 H), 3.26-3.17 (m, 1 H), 2.79-2.76 (m, 1 H), 2.71-2.68 (m, 1 H), 2.56-2.46 (m, 2 H), 2.31 (s, 6 H), 2.00-1.86 (m 2 H), 1.68-1.58 (m, 2 H), 1.35 (d, J = 7.3 Hz, 3 H), 1.15 (t, J = 7.2 Hz, 3 H) M/Z (ES+) Calc.: 489.30 Found: 490.40 (M + H) Analytical HPLC: Method A1 SunFire MS C18 (4.16 × 100 mm) 5 um Retention Time: 11.16 min Chiral HPLC Method C1 CHIRALPAK IA (0.46 × 25 cm) Retention Time: 4.786 min |
| 3 | 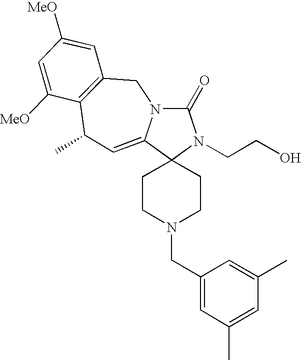 | 819786-01 HCl Salt | NMR ¹H (400 MHz, CDCl₃) δ 12.68 (br s, 1 H), 7.25 (s, 2 H), 7.10 (s, 1 H), 6.42 (d, J = 2.4 Hz, 1 H), 6.41 (d, J = 2.4 Hz, 1 H), 4.79 (d, J = 14.4 Hz, 1 H), 4.75 (d, J = 8.8 Hz, 1 H), 4.61 (d, J = 14.4 Hz, 1 H), 4.05-4.14 (m, 3 H), 3.81 (s, 3 H), 3.79 (s, 3 H), 3.76 (m, 2 H), 3.58 (m, 2 H), 3.48 (d, J = 10.0 Hz, 1 H), 3.35 (d, J = 10.8 Hz, 1 H), 3.11 (q, J = 10 HZ, 2 H), 2.82-3.00 (m, 2 H), 2.37 (s, 6 H), 1.89 (d, J = 14.2 Hz, 1 H), 1.73 (d, 13.9 Hz, 1 H), 1.34 (d, J = 7.2 Hz, 3 H) M/Z (ES+) Calc.: 505.29 Found: 506.40 (M + H) Analytical HPLC: Method A1 SunFire MS C18 (4.6 × 100 mm) 5 um Retention Time: 9.88 min Chiral HPLC Method C1 CHIRALPAK IA (0.46 × 25 cm) Retention Time: 6.687 min |
| 4 | 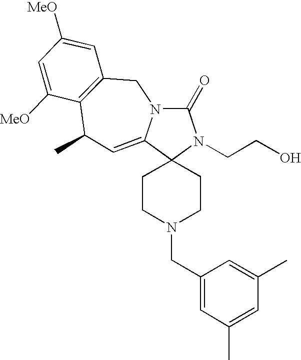 | 819787-01 HCl Salt | NMR ¹H (400 MHz, DMSO) δ 12.67 (br s, 1 H), 7.27 (s, 2 H), 7.11 (s, 1 H), 6.43 (d, J = 2.4 Hz, 1 H), 6.41 (d, J = 2.4 Hz, 1 H), 4.75- 4.81 (m, 2 H), 4.61 (d, J = 14.4 Hz, 1 H), 4.10 (br s, 3 H), 3.81 (s, 3 H), 3.79 (s, 3 H), 3.77 (m, 2 H), 3.58 (br s, 2 H), 3.49 (br s, 1 H), 3.35 (br. s, 1 H), 2.87-3.11 (m, 4 H), 2.37 (s, 6 H), 1.89 (br s, 1 H), 1.73 (d, 11.2 Hz, 1 H), 1.35 (d, J = 5.2 Hz, 3 H) M/Z (ES+) Calc.: 505.29 Found: 506.40 (M + H) Analytical HPLC: Method A1 SunFire MS C18 (4.6 × 100 mm) 5 um Retention Time: 9.87 min Chiral HPLC Method C1 CHIRALPAK IA (0.46 × 25 cm) Retention Time: 4.435 min |
| 5 | 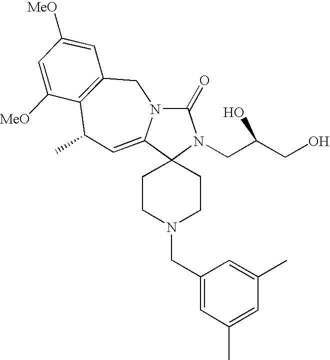 | 819788-01 HCl Salt | NMR ¹H (400 MHz, CDCl₃) δ 12.29 (br s, 1 H), 7.24 (s, 2 H), 7.10 (s, 1 H), 6.43 (d, J = 2.4 Hz, 1 H), 6.41 (d, J = 2.4 Hz, 1 H), 4.80 (d, J = 8.8 Hz, 1 H), 4.78 (d, J = 14.4 Hz, 1 H), 4.64 (d, J = 14.4 Hz, 1 H), 4.04-4.16 (m, 3 H), 3.81 (s, 3 H), 3.79 (s, 3 H), 3.77-3.85 (m, 2 H), 3.61 (d, J = 5.6 Hz, 2 H),3.37- 3.54 (m, 3 H), 2.91-3.11 (m, 4 H), 2.36 (s, 6 H), 1.92 (d, J = 11.6 Hz, 1 H), 1.72 (d, 13.2 Hz, 1 H), 1.37 (d, J = 7.3 Hz, 3 H) M/Z (ES+) Calc.: 535.30 Found: 536.39 (M + H) Analytical HPLC: Method A1 SunFire MS C18 (4.6 × 100 mm) 5 um Retention Time: 9.27 min Chiral HPLC Method C1 CHIRALPAK IA(0.46 × 25 cm) Retention Time: 8.471 min |
| 6 | 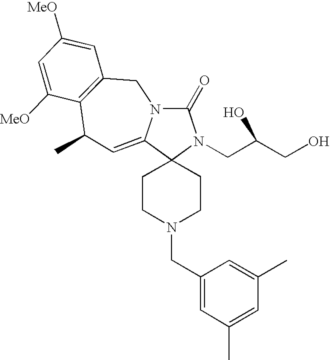 | 819789-01 HCl Salt | NMR ¹H (400 MHz, CDCl₃) δ 12.32 (br s, 1 H), 7.24 (s, 2 H), 7.11 (s, 1 H), 6.44 (d, J = 2.4 Hz, 1 H), 6.42 (d, J = 2.4 Hz, 1 H), 4.80 (d, J = 8.8 Hz, 1 H), 4.80 (d, J = 14.6 Hz, 1 H), 4.63 (d, J = 14.4 Hz, 1 H), 4.05-4.16 (m, 3 H), 3.81 (s, 3 H), 3.80 (s, 3 H), 3.76-3.79 (m, 2 H), 3.60 (d, J = 5.6 Hz,2 H), 3.38- 3.53 (m, 3 H), 2.94-3.08 (m, 4 H), 2.37 (s, 6 H), 1.87 (d, J = 11.2 Hz, 1 H), 1.78 (d, 13.2 Hz, 1 H), 1.36 (d, J = 7.3 Hz, 3 H) M/Z (ES+) Calc.: 535.30 Found: 536.39 (M + H) Analytical HPLC: Method A1 SunFire MS C18 (4.6 × 100 mm) 5 um Retention Time: 9.27 min Chiral HPLC Method C1 CHIRALPAK IA(0.46 × 25 cm) Retention Time: 5.591 min |
| 7 | 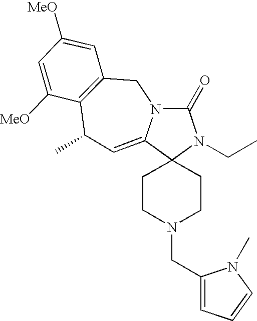 | 819924-01 HCl Salt | NMR ¹H (400 MHz, CD₃OD) δ 6.83-6.81 (m, 1 H), 6.45 (d, J = 2.3 Hz, 1 H), 6.38 (d, J = 2.3 Hz, 2 H), 6.12 (t, J = 3.2 Hz, 1 H), 5.17 (br s, 1 H), 4.67 (d, J = 14.4 Hz, 1 H), 4.59 (d, J = 14.4 Hz, 1 H), 4.44 (s, 2 H), 4.24-4.17 (m, 1 H), 3.74 (t, J = 7.8 Hz, 6 H), 3.62- 3.54 (m, 2 H), 3.46-3.35 (m, 2 H), 2.21 (br s, 2 H), 2.62 (s, 3 H), 2.23-2.14 (m, 2 H), 1.95 (br d, J = 13.8 Hz, 1 H), 1.78 (br d, J = 13.5 Hz, 1 H), 1.35 (d, J = 7.3 Hz, 3 H), 1.13 (t, J = 7.0 Hz, 3 H) M/Z (ES+) Calc.: 464.28 Found: 465.39 (M + H) Analytical HPLC: Method A1 SunFire MS C18 (4.6 × 100 mm) 5 um Retention Time: 9.59 min Chiral HPLC Method C1 CHIRALPAK IA (0.46 × 25 cm) Retention Time: 7.790 min |
| 8 | 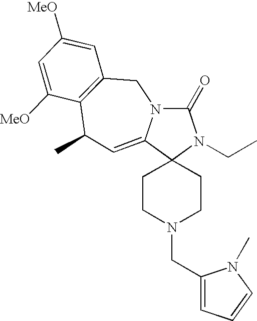 | 819925-00 Salt Free | NMR ¹H (400 MHz, CD₃OD) δ 6.83-6.81 (m, 1 H), 6.45 (d, J = 2.3 Hz, 1 H), 6.38 (d, J = 2.3 Hz, 2 H), 6.11 (t, J = 3.2 Hz, 1 H), 5.17 (br s, 1 H), 4.67 (d, J = 14.4 Hz, 1 H), 4.59 (d, J = 14.4 Hz, 1 H), 4.34 (s, 2 H), 4.24-4.17 (m, 1 H), 3.74 (t, J = 7.9 Hz, 6 H), 3.67- 3.36 (m, 4 H), 3.19 (br s, 2 H), 2.62 (s, 3 H), 2.27-2.14 (m, 2 H), 1.95 (br d, J = 12.6 Hz, 1 H), 1.78 (br d, J = 11.4 Hz, 1 H), 1.35 (d, J = 7.3 Hz, 3 H), 1.13 (t, J = 7.0 Hz, 3 H) M/Z (ES+) Calc.: 464.28 Found: 465.39 (M + H) Analytical HPLC: Method A1 SunFire MS C18 (4.6 × 100 mm) 5 um Retention Time: 9.58 min Chiral HPLC Method C1 CHIRALPAK IA (0.46 × 25 cm) Retention Time: 4.821 min |
| 9 | 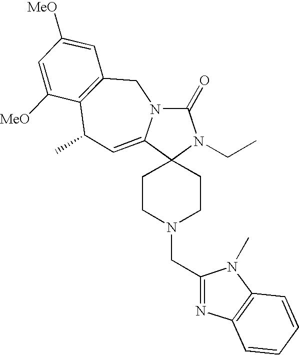 | 819926-01 HCl Salt | NMR ¹H (400 MHz, CD₃OD) δ 7.91-7.87 (m, 1 H), 7.85-7.81 (m, 1 H), 7.67-7.60 (m, 2 H), 6.46 (d, J = 2.3 Hz, 1 H), 6.39 (d, J = 2.3 Hz, 1 H), 4.80 (s, 1 H), 4.68 (d, J = 14.4 Hz, 1 H), 4.60 (d, J = 14.1 Hz, 1 H), 4.23 (q, J = 7.3, 14.6 Hz, 1 H), 4.15 (s, 2 H), 3.76 (s, 3 H), 3.74 (s, 3 H), 3.58-3.41 (m, 4 H), 3.33-3.28 (m, 2 H), 2.62 (s, 3 H), 2.44-2.33 (m, 2 H), 1.93 (br d, J = 16.1 Hz, 1 H), 1.76 (br d, J = 14.4 Hz, 1 H), 1.37 (d, J = 7.3 Hz, 3 H), 1.14 (t, J = 7.0 Hz, 3 H) M/Z (ES+) Calc.: 515.29 Found: 516.36 (M + H) Analytical HPLC: Method A1 SunFire MS C18 (4.6 × 100 mm) 5 um Retention Time: 8.28 min Chiral HPLC Method C1 CHIRALPAK IA (0.46 × 25 cm) Retention Time: 7.461 min |
| 10 | 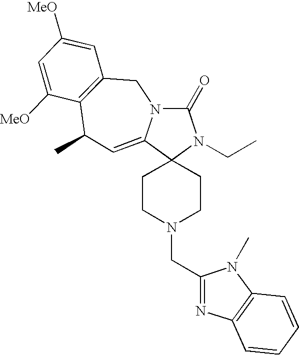 | 819927-01 HCl Salt | NMR ¹H (400 MHz, CD₃OD) δ 7.96-7.92 (m, 1 H), 7.90-7.86 (m, 1 H), 7.71-7.64 (m, 2 H), 6.50 (d, J = 2.3 Hz, 1 H), 6.43 (d, J = 2.3 Hz, 1 H), 4.86 (s, 1 H), 4.72 (d, J = 14.4 Hz, 1 H), 4.64 (d, J = 14.1 Hz, 1 H), 4.27 (q, J = 7.2, 14.5 Hz, 1 H), 4.20 (s, 2 H), 3.80 (s, 3 H), 3.78 (s, 3 H), 3.68-3.39 (m, 4 H), 3.37-3.31 (m, 2 H), 2.66 (s, 3 H), 2.49-2.42 (m, 2 H), 1.98 (br d, J = 14.6 Hz, 1 H), 1.81 (br d, J = 14.4 Hz, 1 H), 1.41 (d, J = 7.3 Hz, 3 H), 1.18 (t, J = 7.0 Hz, 3 H) M/Z (ES+) Calc.: 515.29 Found: 516.36 (M + H) Analytical HPLC: Method A1 SunFire MS C18 (4.6 × 100 mm) 5 um Retention Time: 8.28 min Chiral HPLC Method C1 CHIRALPAK IA (0.46 × 25 cm) Retention Time: 5.670 min |
| 11 | 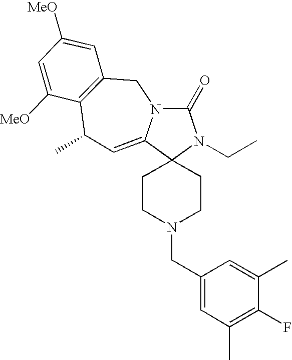 | 819931-00 Salt Free | NMR ¹H (400 MHz, CD₃OD) δ 7.02 (d, J = 7.0 Hz, 2 H), 6.47 (d, J = 2.3 Hz, 1 H), 6.41 (d, J = 2.3 Hz, 1 H), 5.10 (d, J = 8.8 Hz, 1 H), 4.68 (d, J = 14.4 Hz, 1 H), 4.57 (d, J = 14.4 Hz, 1 H), 4.19-4.12 (m, 1 H), 3.79 (s, 3 H), 3.77 (s, 3 H), 3.51 (s, 2 H), 3.28-3.18 (m, 2 H), 2.82-2.73 (m, 2 H), 2.65-2.54 (m, 2 H), 2.24 (d, J = 1.8 Hz, 6 H), 2.07-1.90 (m, 2 H), 1.69 (br d, J = 12.0 Hz, 1 H), 1.54 (br d, J = 13.5 Hz, 1 H), 1.34 (d, J = 7.3 Hz, 3 H), 1.14 (t, J = 7.0 Hz, 3 H) M/Z (ES+) Calc.: 507.29 Found: 508.42 (M + H) Analytical HPLC: Method A1 SunFire MS C18 (4.6 × 100 mm) 5 um Retention Time: 11.22 min Chiral HPLC Method C1 CHIRALPAK IA (0.46 × 25 cm) Retention Time: 6.958 min |
| 12 | 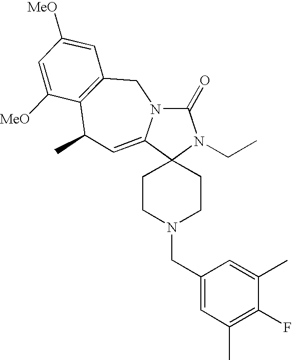 | 819943-00 Salt Free | NMR ¹H (400 MHz, CDCl₃) δ 6.92 (d, J = 7.0 Hz, 2 H), 6.42 (d, J = 2.3 Hz, 1 H), 6.39 (d, J = 2.3 Hz, 1 H), 4.99 (d, J = 8.5 Hz, 1 H), 4.79 (d, J = 14.1 Hz, 1 H), 4.56 (d, J = 14.4 Hz, 1 H), 4.15-4.08 (m, 1 H), 3.77 (d, J = 3.2 Hz, 6 H), 3.42 (s, 2 H), 3.29-3.24 (m, 1 H), 3.24-3.15 (m, 1 H),2.75-2.65 (m, 2 H), 2.53-2.43 (m, 2 H), 2.23 (d, J = 2.1 Hz, 6 H), 1.97-1.83 (m, 2 H), 1.66- 1.56 (m, 2 H), 1.33 (d, J = 7.3 Hz, 3 H), 1.13 (t, J = 7.0 Hz, 3 H) M/Z (ES+) Calc.: 507.29 Found: 508.36 (M + H) Analytical HPLC: Method A1 SunFire MS C18 (4.6 × 100 mm) 5 um Retention Time: 11.20 min Chiral HPLC Methoc C1 CHIRALPAK IA (0.46 × 25 cm) Retention Time: 4.684 min |
| 13 | 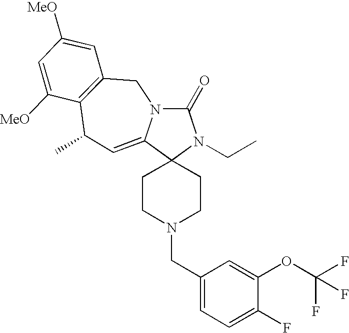 | 819933-00 Salt Free | NMR ¹H (400 MHz, CDCl₃) δ 7.29 (br d, J = 6.7, 1 H), 7.24-7.21 (m, 1 H), 7.12 (t, J = 9.1 Hz, 1 H), 6.42 (d, J = 2.3 Hz, 1 H), 6.39 (d, J = 2.3 Hz, 1 H), 4.98 (d, J = 8.5 Hz, 1 H), 4.79 (d, J = 14.4 Hz, 1 H), 4.56 (d, J = 14.4 Hz, 1 H), 4.15-4.08 (m, 1 H), 3.77 (d, J = 2.3 Hz, 6 H),3.52 (s, 2 H), 3.31-3.24 (m, 1 H), 3.24-3.15 (m, 1 H), 2.73-2.62 (m, 2 H), 2.57-2.48 (m, 2 H), 1.73-1.83 (m, 2 H), 1.68-1.55 (m, 2 H), 1.33 (d, J = 7.0 Hz, 3 H), 1.15 (t, J = 7.0 Hz, 3 H) M/Z (ES+) Calc.: 563.24 Found: 564.30 (M + H) Analytical HPLC: Method A1 SunFire MS C18 (4.6 × 100 mm) 5 um Retention Time: 11.45 min Chiral HPLC Method C1 CHIRALPAK IA (0.46 × 25 cm) Retention Time: 5.872 min |
| 14 | 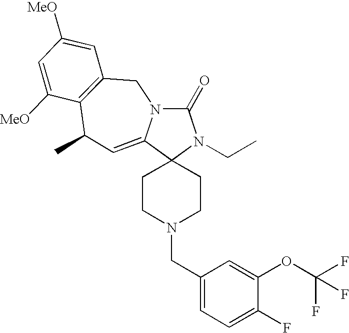 | 819945-00 Salt Free | NMR ¹H (400 MHz, CDCl₃) δ 7.29 (br d, J = 7.3, 1 H), 7.24-7.21 (m, 1 H), 7.12 (t, J = 8.9 Hz, 1 H), 6.42 (d, J = 2.3 Hz, 1 H), 6.39 (d, J = 2.6 Hz, 1 H), 4.98 (d, J = 8.5 Hz, 1 H), 4.79 (d, J = 14.4 Hz, 1 H), 4.56 (d, J = 14.4 Hz, 1 H), 4.15-4.08 (m, 1 H), 3.77 (d, J = 2.1 Hz,6 H), 3.52 (s, 2 H), 3.31-3.24 (m, 1 H), 3.22-3.15 (m, 1 H), 2.73-2.62 (m, 2 H), 2.57-2.48 (m, 2 H), 1.97-1.83 (m, 2 H), 1.68-1.58 (m, 2 H), 1.33 (d, J = 7.30 Hz, 3 H), 1.15 (t, J = 7.0 Hz, 3 H) M/Z (ES+) Calc.: 563.24 Found: 564.30 (M + H) Analytical HPLC: Method A1 SunFire MS C18 (4.6 × 100 mm) 5 um Retention Time: 11.77 min Chiral HPLC Method C1 CHIRALPAK IA (0.46 × 25 cm) Retention Time: 4.144 min |
| 15 | 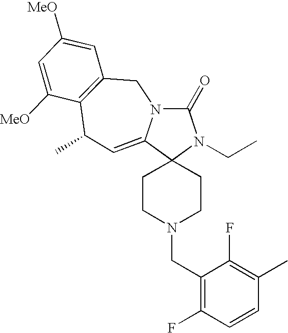 | 819934-00 Salt Free | NMR ¹H (400 MHz, CD₃OD) δ 7.24 (q, J = 8.3, 15.1 Hz, 1 H), 6.91 (t, J = 8.8 Hz, 1 H), 6.47 (d, J = 2.3 Hz, 1 H), 6.41 (d, J = 2.3 Hz, 1 H), 5.00 (d, J = 8.8 Hz, 1 H), 4.67 (d, J = 14.1 Hz, 1 H), 4.57 (d, J = 14.1 Hz, 1 H), 4.14-4.06 (m, 1 H), 3.79 (s, 3 H), 3.78 (s, 3 H), 3.76 (s, 2 H), 3.29-3.17 (m, 2 H), 2.89-2.81 (m, 2 H), 2.75-2.70 (m, 2 H), 2.27 (s, 3 H), 2.06-1.95 (m, 2 H), 1.68 (br d, J = 13.5 Hz, 1 H), 1.56 (br d, J = 13.5 Hz, 1 H), 1.31 (d, J = 7.3 Hz, 3 H), 1.13 (t, J = 7.0 Hz, 3 H) M/Z (ES+) Calc.: 511.26 Found: 512.39 (M + H) Analytical HPLC: Method A1 SunFire MS C18 (4.6 × 100 mm) 5 um Retention Time: 10.52 min Chiral HPLC Method C1 CHIRALPAK IA (0.46 × 25 cm) Retention Time: 6.734 min |
| 16 | 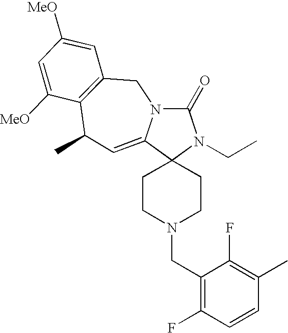 | 819946-00 Salt Free | NMR ¹H (400 MHz, CDCl₃) δ 7.07 (q, J = 8.3, 14.8 Hz, 1 H), 6.77 (t, J = 8.1 Hz, 1 H), 6.41 (d, J = 2.3 Hz, 1 H), 6.38 (d, J = 2.3 Hz, 1 H), 4.89 (d, J = 8.5 Hz, 1 H), 4.78 (d, J = 14.1 Hz, 1 H), 4.54 (d, J = 14.4 Hz, 1 H), 4.10- 4.02 (m, 1 H), 3.77 (d, J = 2.6 Hz, 6 H), 3.68 (s, 2 H),3.29-3.21 (m, 1 H), 3.19- 3.10 (m, 1 H), 2.81-2.71 (m, 2 H), 2.61 (q, J = 12.0, 23.1 Hz, 2 H), 2.23 (s, 3 H), 1.96- 1.83 (m, 2 H), 1.65-1.55 (m, 2 H), 1.30 (d, J = 7.3 Hz, 3 H), 1.10 (t, J = 7.0 Hz, 3 H) M/Z (ES+) Calc.: 511.26 Found: 512.39 (M + H) Analytical HPLC: Method A1 SunFire MS C18 (4.6 × 100 mm) 5 um Retention Time: 10.45 min Chiral HPLC Method C1 CHIRALPAK IA (0.46 × 25 cm) Retention Time: 4.799 min |
| 17 | 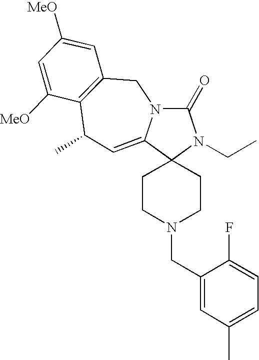 | 819935-00 Salt Free | NMR ¹H (400 MHz, CD₃OD) δ 7.22 (d, J = 7.0 Hz, 1 H), 7.15-7.11 (m, 1 H), 6.98 (t, J = 9.2 Hz, 1 H), 6.48 (d, J = 2.3 Hz, 1 H), 6.41 (d, J = 2.6 Hz, 1 H), 5.05 (d, J = 8.5 Hz, 1 H), 4.68 (d, J = 14.4 Hz, 1 H), 4.57 (d, J = 14.4 Hz, 1 H), 4.16-4.09 (m, 1 H), 3.79 (s, 3 H), 3.77 (s, 3 H), 3.66 (s, 2 H),3.28-3.20 (m, 2 H), 2.87-2.79 (m, 2 H), 2.72-2.63 (m, 2 H), 2.33 (s, 3 H), 2.06-1.94 (m, 2 H), 1.69 (br d, J = 13.5 Hz, 1 H), 1.56 (br d, J = 13.5 Hz, 1 H), 1.32 (d, J = 7.3 Hz, 3 H), 1.14 (t, J = 7.0 Hz, 3 H) M/Z (ES+) Calc.: 493.27 Found: 494.37 (M + H) Analytical HPLC: Method A1 SunFire MS C18 (4.6 × 100 mm) 5 um Retention Time: 10.48 Chiral HPLC Method C1 CHIRALPAK IA (0.46 × 25 cm) Retention Time: 6.585 min |
| 18 | 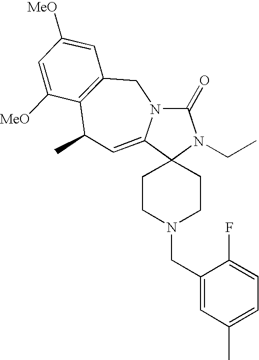 | 819947-00 Salt Free | NMR ¹H (400 MHz, CD₃OD) δ 7.23-7.21 (m, 1 H), 7.15-7.11 (m, 1 H), 7.98 (t, J = 9.2 Hz, 1 H), 6.47 (d, J = 2.3 Hz, 1 H), 6.41 (d, J = 2.3 Hz, 1 H), 5.05 (d, J = 8.8 Hz, 1 H), 4.68 (d, J = 14.4 Hz, 1 H), 4.57 (d, J = 14.4 Hz, 1 H), 4.16-4.09 (m, 1 H), 3.79 (s, 3 H), 3.77 (s, 3 H), 3.66 (s, 2 H), 3.28-3.18 (m, 2 H), 2.87-2.79 (m, 2 H), 2.72-2.61 (m, 2 H), 2.33 (s, 3 H), 2.06-1.94 (m, 2 H), 1.69 (br d, J = 11.7 Hz, 1 H), 1.56 (br d, J = 13.8 Hz, 1 H), 1.32 (d, J = 7.3 Hz, 3 H), 1.14 (t, J = 7.0 Hz, 3 H) M/Z (ES+) Calc.: 493.27 Found: 494.41 (M + H) Analytical HPLC: Method A1 SunFire MS C18 (4.6 × 100 mm) 5 um Retention Time: 10.52 min Chiral HPLC Method C1 CHIRALPAK IA (0.46 × 25 cm) Retention Time: 4.695 min |
| 19 | 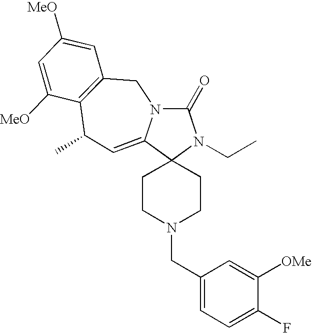 | 819936-00 Salt Free | NMR ¹H (400 MHz, CD₃OD) δ 7.09-7.07 (m, 1 H), 7.02-6.97 (m, 1 H), 6.88-6.84 (m, 1 H), 6.44 (d, J = 2.3 Hz, 1 H), 6.38 (d, J = 2.6 Hz, 1 H), 5.10 (d, J = 8.8 Hz, 1 H), 4.65 (d, J = 14.4 Hz, 1 H), 4.55 (d, J = 14.4 Hz, 1 H), 4.16-4.09 (m, 1 H), 3.84 (s, 3 H), 3.75 (d, J = 9.1 Hz, 6 H), 3.55 (s, 2 H), 3.26-3.19 (m, 2 H), 2.81-.272 (m, 2 H), 2.63-2.527 (m, 2 H), 2.03-1.90 (m, 2 H), 1.67 (br d, J = 13.5 Hz, 1 H), 1.54 (br d, J = 13.5 Hz, 1 H), 1.31 (d, J = 7.3 Hz, 3 H), 1.11 (t, J = 7.0 Hz, 3 H) M/Z (ES+) Calc.: 509.27 Found: 510.47 (M + H) Analytical HPLC: Method A1 SunFire MS C18 (4.6 × 100 mm) 5 um Retention Time: 9.71 min Chiral HPLC Method C1 CHIRALPAK IA (0.46 × 25 cm) Retention Time: 6.902 min |
| 20 | 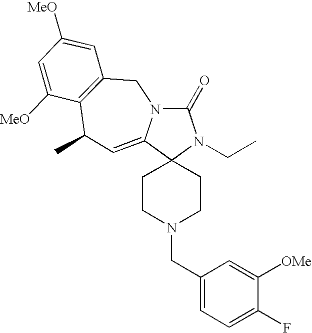 | 819948-00 Salt Free | NMR ¹H (400 MHz, CD₃OD) δ 7.13-7.10 (m, 1 H), 7.06-7.01 (m, 1 H), 6.91-6.88 (m, 1 H), 6.48 (d, J = 2.6 Hz, 1 H), 6.41 (d, J = 2.6 Hz, 1 H), 5.13 (d, J = 8.8 Hz, 1 H), 4.69 (d, J = 14.4 Hz, 1 H), 4.58 (d, J = 14.1 Hz, 1 H), 4.20-4.13 (m, 1 H), 3.88 (s, 3 H), 3.80 (s, 3 H), 3.77 (s, 3 H), 3.58 (s, 2 H), 3.28-3.20 (m, 2 H), 2.85-2.75 (m, 2 H), 2.67-2.56 (m, 2 H), 2.06-1.93 (m, 2 H), 1.71 (br d, J = 11.7 Hz, 1 H), 1.57 (br d, J = 11.7 Hz, 1 H), 1.35 (d, J = 7.0 Hz, 3 H), 1.15 (t, J = 7.0 Hz, 3 H) M/Z (ES+) Calc.: 509.27 Found: 510.47 (M + H) Analytical HPLC: Method A1 SunFire MS C18 (4.6 × 100 mm) 5 um Retention Time: 9.70 min Chiral HPLC Method C1 CHIRALPAK IA (0.46 × 25 cm) Retention Time: 4.781 min |
| 21 | 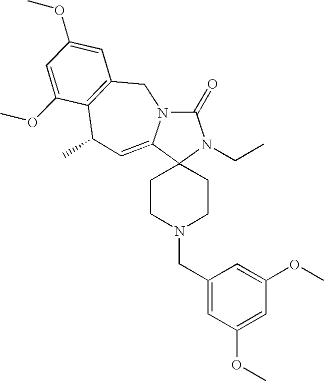 | 829893-00 Salt Free | NMR ¹H (400 MHz, CD₃OD) δ 6.54 (d, J = 2.1 Hz, 2 H), 6.48 (d, J = 2.3 Hz, 1 H), 6.42-6.40 (m, 2 H), 5.12 (d, J = 8.5 Hz, 1 H), 4.68 (d, J = 14.1 Hz, 1 H), 4.58 (d, J = 14.1 Hz, 1 H), 4.20-4.12 (m, 1 H), 3.80 (s, 3 H), 3.78 (d, J = 0.9 Hz, 9 H), 3.56 (s, 2 H), 3.29-3.20 (m, 2 H), 2.85-2.77 (m, 2 H), 2.67-2.56 (m, 2 H), 2.07-1.95 (m, 2 H), 1.72-1.68 (m, 1 H), 1.58-1.55 (m, 1 H), 1.34 (d, J = 7.3 Hz, 3 H), 1.15 (t, J = 7.0 Hz, 3 H) M/Z (ES+) Calc.: 521.29 Found: 522.39 (M + H) Analytical HPLC: Method A1 SunFire MS C18 (4.6 × 100 mm) 5 um Retention Time: 10.22 min Chiral HPLC Method C1 CHIRALPAK IA (0.46 × 25 cm) Retention Time: 8.084 min |
| 22 | 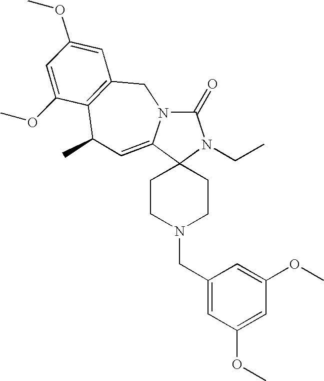 | 819950-00 Salt Free | NMR ¹H (400 MHz, CDCl₃) δ 6.49 (d, J = 2.3 Hz, 2 H), 6.42 (d, J = 2.6 Hz, 1 H), 6.39 (d, J = 2.6 Hz, 1 H), 6.34 (t, J = 2.2 Hz, 1 H), 5.00 (d, J = 8.5 Hz, 1 H), 4.79 (d, J = 14.4 Hz, 1 H), 4.56 (d, J = 14.4 Hz, 1 H), 4.15- 4.08 (m, 1 H), 3.78 (s, 6 H), 3.77 (d, J = 2.1 Hz, 6 H), 3.49(s, 2 H), 3.33-3.24 (m ,1 H), 3.24-3.15 (m, 1 H), 2.77-2.67 (m, 2 H), 2.55-2.46 (m, 2 H), 1.99-1.85 (m, 2 H), 1.66-1.57 (m, 2 H), 1.33 (d, J = 7.0 Hz, 3 H), 1.14 (t, J = 7.0 Hz, 3 H) M/Z (ES+) Calc.: 521.29 Found: 522.38 (M + H) Analytical HPLC: Method A1 SunFire MS C18 (4.6 × 100 mm) 5 um Retention Time: 10.25 min Chiral HPLC Method C1 CHIRALPAK IA (0.46 × 25 cm) Retention Time: 5.601 min |
| 23 | 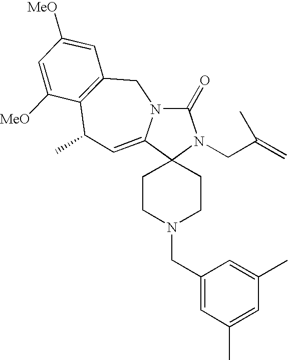 | 820006-01 HCl Salt | NMR ¹H (400 MHz, DMSO) δ 12.85 (br s, 1 H), 7.24 (s, 2 H), 7.10 (s, 1 H), 6.40-6.42 (m, 2 H), 4.79-4.85 (m, 3 H), 4.54-4.67 (m, 2 H), 3.86-4.14 (m, 5 H), 3.80 (s, 3 H), 3.79 (s, 3 H), 3.30-3.49 (m, 2 H), 2.82- 3.14 (m, 4 H), 2.37 (s, 6 H), 1.80 (d, J = 13.7 Hz, 1 H), 1.76 (s, 3 H), 1.68 (d, 14.2 Hz, 1 H), 1.34 (d, J = 7.1 Hz, 3 H) M/Z (ES+) Calc.: 515.31 Found: 516.42 (M + H) Analytical HPLC: Method A1 SunFire MS C18 (4.6 × 100 mm) 5 um Retention Time: 12.04 min Chiral HPLC Method C1 CHIRALPAK IA (0.46 × 25 cm) Retention Time: 7.576 min |
| 24 | 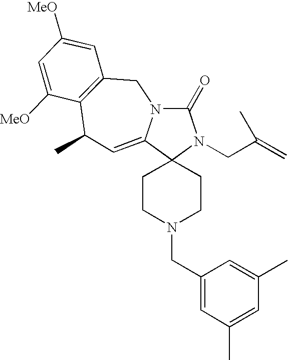 | 820007-01 HCl Salt | NMR ¹H (400 MHz, DMSO) δ 12.84 (br s, 1 H), 7.24 (s, 2 H), 7.10 (s, 1 H), 6.41-6.42 (m, 2 H), 4.79 -4.85 (m, 3 H), 4.54-4.67 (m, 2 H), 3.86-4.14 (m, 5 H), 3.80 (s, 3 H), 3.79 (s, 3 H), 3.29-3.46 (m, 2 H), 2.82- 3.14 (m, 4 H), 2.36 (s, 6 H), 1.80 (d, J = 13.9 Hz, 1 H), 1.76 (s, 3 H), 1.69 (d, 14.4 Hz, 1 H), 1.34 (d, J = 7.1 Hz, 3 H) M/Z (ES+) Calc.: 515.31 Found: 516.42 (M + H) Analytical HPLC: Method A1 SunFire MS C18 (4.6 × 100 mm) 5 um Retention Time: 12.02 min Chiral HPLC Method C1 CHIRALPAK IA (0.46 × 25 cm) Retention Time: 5.074 min |
| 25 | 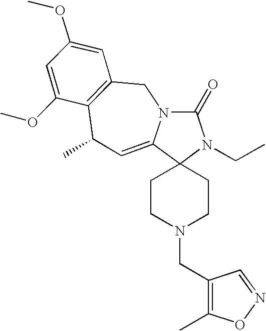 | 819810-01 HCl Salt | NMR ¹H (400 MHz, CD₃OD) δ 8.34 (s, 1 H), 6.46 (d, J = 2.6 Hz, 1 H), 6.41 (d, J = 2.3 Hz, 1 H), 5.13 (d, J = 8.8 Hz, 1 H), 4.69 (d, J = 14.4 Hz, 1 H), 4.58 (d, J = 14.4 Hz, 1 H), 4.21-4.14 (m, 1 H), 3.78 (s, 3 H), 3.76 (s, 3 H), 3.58 (d, J = 2.3 Hz, 2 H), 3.30-3.16 (m, 2 H), 2.93 (br d, J = 12.0 Hz, 1 H), 2.84 (br d, J = 11.1 Hz, 1 H), 2.79-2.65 (m, 2 H), 2.44 (s, 3 H), 2.08-1.94 (m, 2 H), 1.75 (br d, J = 13.8 Hz, 1 H), 1.59 (br d, J = 11.1 Hz, 1 H), 1.34 (d, J = 7.3 Hz, 3 H), 1.14 (t, J = 7.0 Hz, 3 H) M/Z (ES+) Calc.: 466.26 Found: 568.45 (M + H + 101) Analytical HPLC: Method A1 SunFire MS C18(4.6 × 100 mm) 5 um Retention Time: 8.00 min |
| 26 | 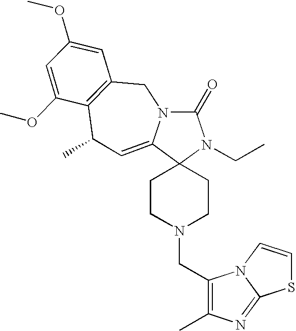 | 811352-00 Salt Free | NMR ¹H (400 MHz, DMSO) δ 7.82 (d, J = 4.4 Hz, 1 H), 7.16 (d, J = 4.7 Hz, 1 H), 6.49 (d, J = 2.3 Hz, 1 H), 6.39 (d, J = 2.3 Hz, 1 H), 5.06 (d, J = 8.5 Hz, 1 H), 4.64 (d, J = 14.4 Hz, 1 H), 4.50 (d, J = 14.4 Hz, 1 H), 4.10- 4.02 (m, 1 H), 3.76 (s, 3 H), 3.74 (s, 3 H), 3.31 (s, 2 H), 3.17-3.07 (m, 2 H), 2.76- 2.52 (m, 4 H), 2.21 (s, 3 H), 1.86-1.72 (m, 2 H), 1.56 (br d, J = 11.1 Hz, 1 H), 1.40 (br d, J = 12.3 Hz, 1 H), 1.28 (d, J = 7.3 Hz, 3 H), 1.03 (t, J = 6.9 Hz, 3 H) M/Z (ES+) Calc.: 521.25 Found: 522.34 (M + H) Analytical HPLC: Method A1 SunFire MS C18 (4.6 × 100 mm) 5 um Retention Time: 8.05 min |
| 27 | 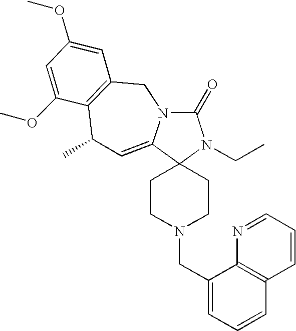 | 819955-01 HCl Salt | (400 MHz, CD₃OD) δ 8.93-8.92 (m, 1 H), 8.36-8.33 (m, 1 H), 7.91-7.86 (m, 2 H), 7.61 (t, J = 7.6 Hz, 1 H), 7.55-7.52 (m, 1 H), 6.48 (d, J = 2.3 Hz, 1 H), 6.41 (d, J = 2.3 Hz, 1 H), 5.17 (d, J = 8.5 Hz, 1 H), 4.68 (d, J = 14.4 Hz, 1 H), 4.58 (d, J = 14.4 Hz, 1 H), 4.32 (s, 2 H), 4.20-4.12 (m, 1 H), 3.80 (s, 3 H), 3.77 (s, 3 H), 3.30-3.19 (m, 2 H), 2.99-2.86 (m, 2 H), 2.86-2.74 (m, 2 H), 2.10-1.97 (m, 2 H), 1.69 (br d, J = 13.5 Hz, 1 H), 1.56 (br d, J = 11.7 Hz, 1 H), 1.34 (d, J = 7.3 Hz, 3 H), 1.13 (t, J = 7.0 Hz, 3 H) M/Z (ES+) Calc.: 512.28 Found: 513.40 (M + H) Analytical HPLC: Method A1 SunFire MS C18 (4.6 × 100 mm) 5 um Retention Time: 9.80 min |
| 28 | 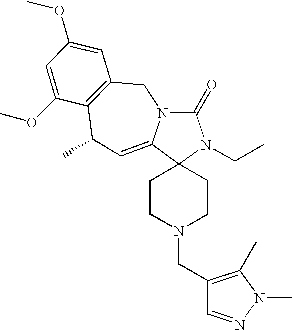 | 819976-01 HCl Salt | (500 MHz, CD₃OD) δ 7.60 (s, 1 H), 6.50 (d, J = 2.3 Hz, 1 H), 6.43 (d, J = 2.3 Hz, 1 H), 5.22 (br s, 1 H), 4.71 (d, J = 14.2 Hz, 1 H), 4.64 (d, J = 14.2 Hz, 1 H), 4.30 (br s, 1 H), 4.25 (br s, 1 H), 3.83-3.77 (m, 9 H), 3.57 (br s, 1 H), 3.44 (br s, 2 H), 3.22 (br s, 2 H), 2.65 (s, 2 H), 2.39 (s, 3 H), 2.26- 2.13(m, 2 H), 2.00 (br d, J = 11.4 Hz, 1 H), 1.82 (br d, J = 13.3 Hz, 1 H), 1.39 (d, J = 6.9 Hz, 3 H), 1.17 (t, J = 6.9 Hz, 3 H) M/Z (ES+) Calc.: 479.29 Found: 480.45 (M + H) Analytical HPLC: Method A1 SunFire MS C18 (4.6 × 100 mm) 5 um Retention Time: 7.22 min |
| 29 | 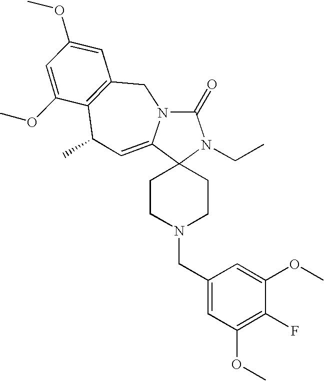 | 824214-00 Salt Free | (400 MHz, CDCl₃) δ 6.60 (d, J = 7.0 Hz, 2 H), 6.45 (d, J = 2.6 Hz, 1 H), 6.42 (d, J = 2.3 Hz, 1 H), 5.03 (d, J = 8.8 Hz, 1 H), 4.82 (d, J = 14.1 Hz, 1 H), 4.59 (d, J = 14.4 Hz, 1 H), 4.18-4.10 (m, 1 H), 3.90 (d, J = 0.9 Hz, 6 H), 3.80 (d, J = 2.9 Hz, 6 H), 3.49 (s, 2 H), 3.35-3.18 (m, 2 H), 2.78- 2.67 (m, 2 H), 2.58-2.47 (m, 2 H), 2.01- 1.86 (m, 2 H), 1.70-1.60 (m, 2 H), 1.37 (d, J = 7.3 Hz, 3 H), 1.18 (t, J = 7.0 Hz, 3 H) M/Z (ES+) Calc.: 539.28 Found: 540.36 (M + H) Analytical HPLC: Method A1 SunFire MS C18 (4.6 × 100 mm) 5 um Retention Time: 9.52 min |
| 30 | 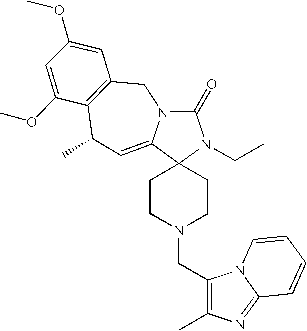 | 811305-01 HCl Salt | (400 MHz, DMSO) δ 9.26 (br s, 1 H), 7.90 (br s, 2 H), 7.51 (br s, 1 H), 6.52 (d, J = 2.1 Hz, 1 H), 6.43 (d, J = 2.3 Hz, 1 H), 4.97 (br s, 1 H), 4.66 (d, J = 14.6 Hz, 1 H), 4.55 (d, J = 14.1 Hz, 1 H), 4.19-4.11 (m, 1 H), 3.79-3.72 (m, 6 H), 3.47 (br s, 6 H), 3.21 (br d, J = 5.9 Hz, 1 H), 2.85 (br s, 1 H), 2.67 (s, 2 H), 2.54 (s, 3 H), 1.79 (br d, J = 13.5 Hz, 1 H), 1.59 (br d, J = 13.2 Hz, 1 H), 1.36 (d, J = 7.3 Hz, 3 H), 1.01 (t, J = 7.0 Hz, 3 H) M/Z (ES+) Calc.: 515.29 Found 516.43 (M + H) Analytical HPLC: Method A1 SunFire MS C18 (4.6 × 100 mm) 5 um Retention Time: 8.23 min |
| 31 | 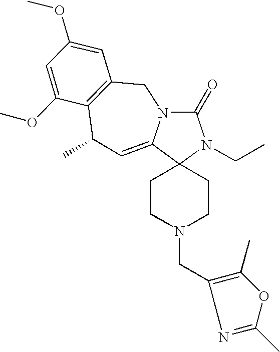 | 811283-01 HCl Salt | M/Z (ES+) Calc.: 480.27 Found: 481.34 (M + H) Analytical HPLC: Method A1 SunFire MS C18 (4.6 × 100 mm) 5 um Retention Time: 7.77 min |
| 32 | 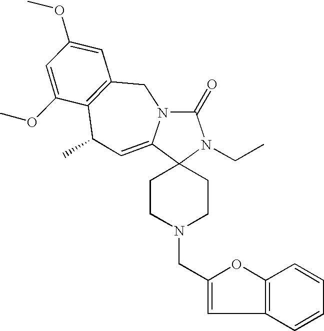 | 811308-01 HCl Salt | (500 MHz, DMSO) δ 7.59 (d, J = 6.9 Hz, 1 H), 7.54 (d, J = 7.3 Hz, 1 H), 7.29-7.25 (m, 1 H), 7.23-7.20 (m, 1 H), 6.80 (s, 1 H), 6.48 (d, J = 2.3 Hz, 1 H), 6.39 (d, J = 2.3 Hz, 1 H), 4.99 (d, J = 8.7 Hz, 1 H), 4.64 (d, J = 14.6 Hz, 1 H), 4.50 (d, J = 14.2 Hz, 1 H), 4.04-3.97 (m, 1 H), 3.76 (s, 3 H), 3.75 (s, 3 H), 3.73 (s, 2 H), 3.20-3.09 (m, 2 H), 2.84-2.59 (m, 4 H), 1.92-1.82 (m, 2 H), 1.58 (br d, J = 13.3 Hz, 1 H), 1.44 (br d, J = 13.3 Hz, 1 H), 1.24 (d, J = 7.3 Hz, 3 H), 1.03 (t, J = 3.0 Hz, 3 H) M/Z (ES+) Calc.: 501.26 Found: 603.38 (M + H + 101) Analytical HPLC: Method A1 SunFire MS C18 (4.6 × 100 mm) 5 um Retention Time: 10.12 min |
| 33 | 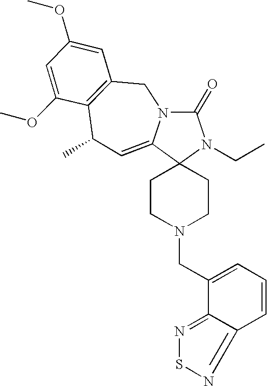 | 811332-01 HCl Salt | (500 MHz, CD₃OD) δ 8.20 (d, J = 9.2 Hz, 1 H), 7.93 (d, J = 6.4 Hz, 1 H), 7.82-7.79 (m, 1 H), 6.50 (d, J = 2.3 Hz, 1 H), 6.43 (d, J = 2.3 Hz, 1 H), 5.30 (br s, 1 H), 4.96 (br s, 2 H), 4.71 (d, J = 14.6 Hz, 1 H), 4.64 (d, J = 14.6 Hz, 1 H), 4.27 (br s, 1 H), 3.81 (s, 3 H), 3.78 (s, 3 H), 3.66 (br s, 4 H), 3.19 (br s, 2 H), 2.21 (br s, 2 H), 1.99 (br s, 1 H), 1.82 (br s, 1 H), 1.40 (br d, J = 6.4 Hz, 3 H), 1.15 (br s, 3 H) M/Z (ES+) Calc.: 519.23 Found: 621.40 (M + H + 101) Analytical HPLC: Method A1 SunFire MS C18 (4.6 × 100 mm) 5 um Retention Time: 10.32 min |
| 34 | 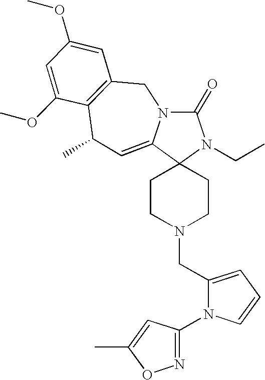 | 820017-01 HCl Salt | (500 MHz, CD₃OD) δ 7.42 (br s, 1 H), 6.67 (br s, 1 H), 6.65 (s, 1 H), 6.50 (d, J = 2.3 Hz, 1 H), 6.43 (d, J = 2.7 Hz, 2 H), 5.30 (br s, 1 H), 4.73-4.63 (m, 3 H), 4.28 (br s, 1 H), 3.80 (s, 3 H), 3.78 (s, 3 H), 3.57 (br s, 3 H), 3.20 (br s, 2 H), 2.65 (s, 3 H), 2.51 (s, 2 H), 2.19 (br s, 2 H), 2.01 (br s, 1 H), 1.84 (br s, 1 H), 1.41 (br d, J = 6.9 Hz, 3 H), 1.20-1.12 (m, 3 H) M/Z (ES+) Calc.: 531.28 Found: 532.42 (M + H) Analytical HPLC: Method A1 SunFire MS C18 (4.6 × 100 mm) 5 um Retention Time: 10.27 min |
| 36 | 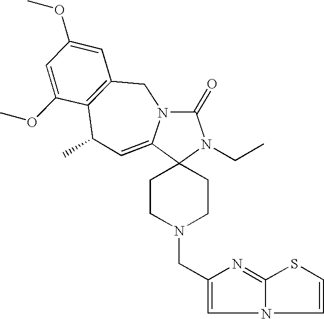 | 811309-01 HCl Salt | (400 MHz, CD₃OD) δ 8.22 (s, 1 H), 8.04- 8.03 (m, 1 H), 7.53-7.51 (m, 1 H), 6.49 (d, J = 2.6 Hz, 1 H), 6.42 (d, J = 2.3 Hz, 1 H), 4.76-4.55 (m, 3 H), 4.26-4.21 (m, 1 H), 3.82-3.76 (m, 8 H), 3.67 (br s, 2 H), 3.59 (d, J = 6.7 Hz, 2 H), 3.29 (br s, 2 H), 2.48- 2.32 (m, 2 H), 2.02 (br s, 1 H), 1.86 (br s, 1 H), 1.38 (d, J = 7.3 Hz, 3 H), 1.17 (t, J = 7.0 Hz, 3 H) M/Z (ES+) Calc.: 507.23 Found: 508.36 (M + H) Analytical HPLC: Method A1 SunFire MS C18 (4.6 × 100 mm) 5 um Retention Time: 6.95 min |
| 37 | 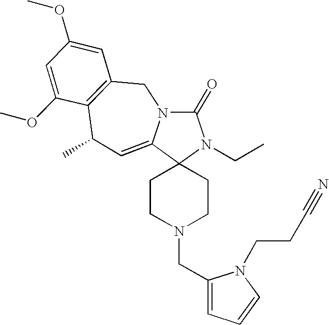 | 820008-00 Salt Free | (400 MHz, CDCl₃) δ 6.72-6.70 (m, 1 H), 6.45 (d, J = 2.3 Hz, 1 H), 6.42 (d, J = 2.3 Hz, 1 H), 6.09 (t, J = 3.2 Hz, 1 H), 6.05- 6.04 (m, 1 H), 5.02 (d, J = 8.8 Hz, 1 H), 4.82 (d, J = 14.4 Hz, 1 H), 4.59 (d, J = 14.4 Hz, 1 H), 4.25 (t, J = 7.2 Hz, 2 H), 4.20- 4.12 (m, 1 H), 3.80 (d, J = 2.9 Hz, 6 H), 3.52 (s, 2 H), 3.31-3.22 (m, 1 H), 3.22- 3.13 (m, 1 H), 2.94 (t, J = 7.0 Hz, 2 H), 2.79-2.68 (m, 2 H), 2.56-2.46 (m, 2 H), 1.91-1.77 (m, 2 H), 1.73-1.62 (m, 2 H), 1.37 (d, J = 7.3 Hz, 3 H), 1.17 (t, J = 7.0 Hz, 3 H) M/Z (ES+) Calc.: 503.29 Found: 504.39 (M + H) Analytical HPLC: Method A1 SunFireMS C18 (4.6 × 100 mm) 5 um Retention Time: 9.56 min |
| 38 | 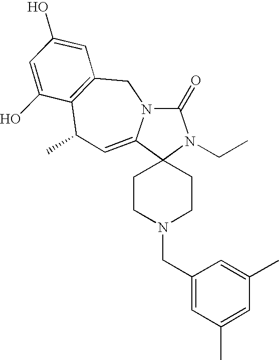 | 819888-00 Salt Free | (400 MHz, CDCl₃) δ 6.94 (s, 2 H), 6.91 (s, 1 H), 6.60 (s, 1 H), 6.28 (s, 1 H), 5.05 (d, J = 8.3 Hz, 1 H), 4.81 (d, J = 14.2 Hz, 1 H), 4.59 (d, J = 14.2 Hz, 1 H), 4.11-4.03 (m, 1 H), 3.53 (s, 2 H), 3.28-3.17 (m, 2 H), 2.79-2.74 (m, 2 H), 2.56-2.53 (m, 2 H), 2.30 (s, 6 H), 1.96-1.90 (m, 2 H), 1.70- 1.59 (m, 2 H), 1.40 (d, J = 7.1 Hz, 3 H), 1.11 (t, J = 6.8 Hz, 3 H) M/Z (ES+) Calc.: 461.27 Found: 462.37 (M + H) Analytical HPLC: Method A1 SunFire MS C18 (4.6 × 100 mm) 5 um Retention Time: 6.57 min |
| 39 | 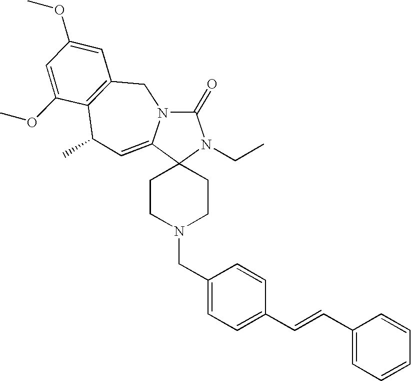 | 819814-01 HCl Salt | (400 MHz, CD₃OD) δ 7.71 (d, J = 7.9 Hz, 2 H), 7.58 (d, J = 8.5 Hz, 4 H), 7.36 (t, J = 7.6 Hz, 2 H), 7.32-7.20 (m, 3 H), 6.49 (d, J = 2.3 Hz, 1 H), 6.42 (d, J = 2.6 Hz, 1 H), 5.23 (d, J = 8.8 Hz, 1 H), 4.71 (d, J = 14.4 Hz, 1 H), 4.63 (d, J = 14.4 Hz, 1 H), 4.46 (s, 2 H), 4.29-4.22 (m, 1 H), 3.79 (s, 3 H), 3.77 (s, 3 H), 3.61-3.47 (m, 4 H), 3.28-3.15 (m, 2 H), 2.35-2.17 (m, 2 H), 1.99 (br d, J = 13.8 Hz, 1 H), 1.80 (br d, J = 14.1 Hz, 1 H), 1.40 (d, J = 7.3 Hz, 3 H), 1.16 (t, J = 7.0 Hz, 3 H) M/Z (ES+) Calc.: 563.31 Found: 564.36 (M + H) Analytical HPLC: Method A1 SunFire MS C18 (4.6 × 100 mm) 5 um Retention Time: 12.04 min |
| 40 | 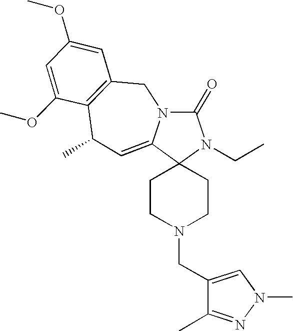 | 819971-00 Salt Free | (400 MHz, CD₃OD) δ 7.46 (s, 1 H), 6.46 (d, J = 2 Hz, 1 H), 6.40 (d, J = 2 Hz, 1 H), 5.07 (d, J = 7 Hz, 1 H), 4.68 (d, J = 14 Hz, 1 H), 4.56 (d, J = 14 Hz, 1 H), 4.10-4.05 (m, 1 H), 3.80-3.72 (m, 9 H), 3.43 (br s, 2 H), 3.22-3.17 (m, 2 H), 2.68-2.48 (m, 2 H), 2.20 (s, 3 H), 2.03-1.86 (m, 2 H), 1.75-1.50 (m, 2 H), 1.33 (d, J = 7 Hz, 3 H), 1.12 (t, J = 6.9 Hz, 3 H) M/Z (ES+) Calc.: 479.29 Found: 480.45 (M + H) Analytical HPLC: Method A1 SunFire MS C18 (4.6 × 100 mm) 5 um Retention Time: 7.15 min |
| 41 | 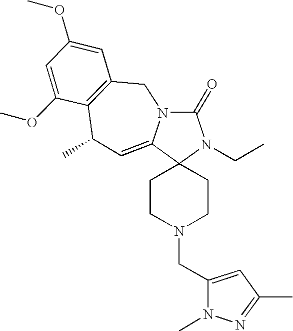 | 819973-00 Salt Free | (400 MHz, CD₃OD) δ 6.45 (d, J = 2 Hz, 1 H), 6.40 (d, J = 2 Hz, 1 H), 5.95 (s, 1 H), 5.11 (d, J = 7 Hz, 1 H), 4.68 (d, J = 14 Hz, 1 H), 4.55 (d, J = 14 Hz, 1 H), 4.22-4.10 (m, 1 H), 3.80-3.69 (m, 9 H), 3.60-6.49 (m, 2 H), 3.30-3.12 (m, 2 H), 2.84-2.49 (m, 2 H), 2.15 (s, 3 H), 2.02-1.83 (m, 2 H), 1.70-1.42 (m, 2 H), 1.33 (d, J = 7 Hz, 3 H), 1.10 (t, J = 6.9 Hz, 3 H) M/Z ES(+) Calc.: 479.29 Found: 480.45 (M + H) Analytical HPLC: Method A1 SunFire MS C18 (4.6 × 100 mm) 5 um Retention Time: 7.98 min |

### Analytical Methods:

### Method A1

Solvent A: 0.2% Et₃N in water

Solvent B: 0.2% Et₃N in acetonitrile

Flow rate: 2.0 ml/min

Linear Gradient:

| time | | |
|---|---|---|
| (min) | % A | % B |
| 0 | 70 | 30 |
| 2 | 70 | 30 |
| 9 | 5 | 95 |
| 14 | 5 | 95 |

### Method C1

Mobile Phase: 0.1% Et₂NH in ethanol

Flow rate: 1.0 ml/min

Isocratic.

### Examples 42-126

### In Vitro Biological Activity

HEKT-bet-luc assay: This assay measures a T-bet dependent reporter (luciferase) activity in engineered HEK cells that express a human T-bet and a T-box responsive element driving luciferase reporter. HEKT-bet cells were plated at 2×104/well in 96-well plate and compound was added into cell culture for 24 hours. Luciferase activity was measured by adding 50 μl of Steady-Glo reagent (Promega) and samples were read in Victor V reader (PerkinElmer). The activity of compound was determined by comparing compound treated samples to non-compound treated vehicle controls. The IC₅₀ values were calculated utilizing a maximum value corresponding to the amount of luciferase in the absence of a test compound and a minimum value corresponding to a test compound value obtained at maximum inhibition.

Determination of Normalized HEKT-bet IC50 values: Compounds were assayed in microtiter plates. Each plate included a reference compound which was ER-819544. The un-normalized IC₅₀ value for a particular compound was divided by the IC₅₀ value determined for the reference compound in the same microtiter plate to provide a relative potency value. The relative potency value was then multiplied by the established potency of the reference compound to provide the normalized HEKT-bet IC₅₀ value. In this assay, the established potency for ER-819544 was 0.035 μM. The IC₅₀ values provided herein were obtained using this normalization method.

Exemplary compounds of the present invention were assayed according to the methods set forth above in the HEKT-bet-luc assay described above. Table 2 below set forth exemplary compounds of the present invention having an IC₅₀ of up to the indicated amount (μM) as determined by the normalized HEKT-bet-luc assay described above.

TABLE 2
IC₅₀ Values of Exemplary Compounds

| Example | | | |
|---|---|---|---|
| # | Structure | ER-Number | IC₅₀ (μm) |
| 42 | 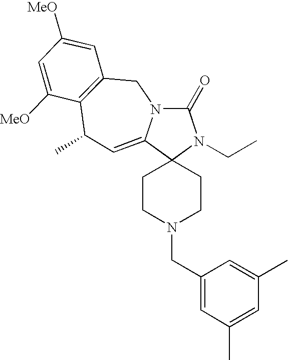 | ER-819762 | 0.04 |
| 43 | 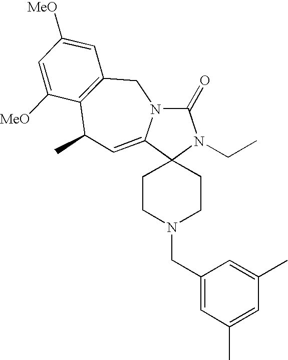 | ER-819763 | 0.55 |
| 44 | 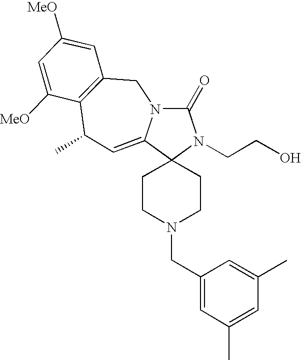 | ER-819786 | 0.03 |
| 45 | 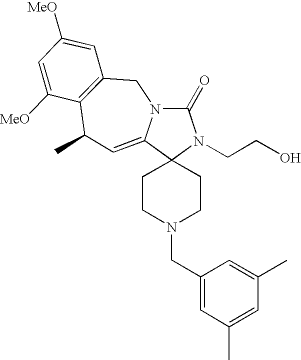 | ER-819787 | 0.17 |
| 46 | 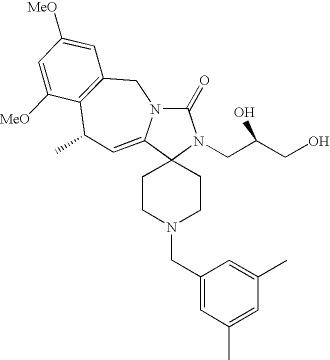 | ER-819788 | 0.03 |
| 47 | 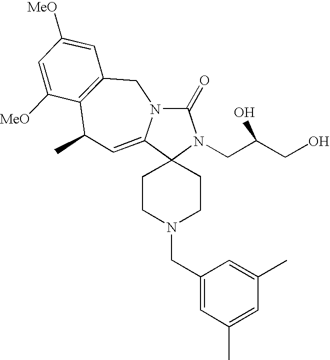 | ER-819789 | 0.17 |
| 48 | 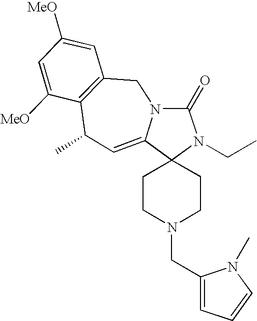 | ER-819924 | 0.05 |
| 49 | 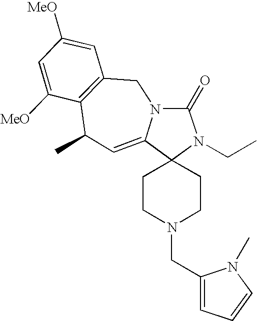 | ER-819925 | 0.32 |
| 50 | 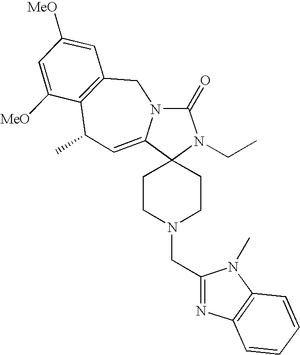 | ER-819926 | 0.04 |
| 51 | 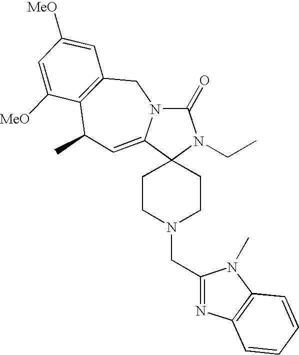 | ER-819927 | 0.21 |
| 52 | 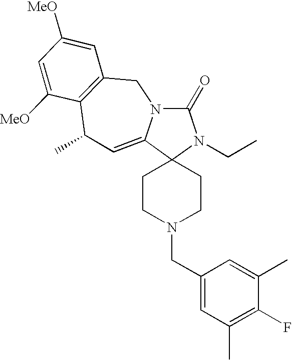 | ER-819931 | 0.07 |
| 53 | 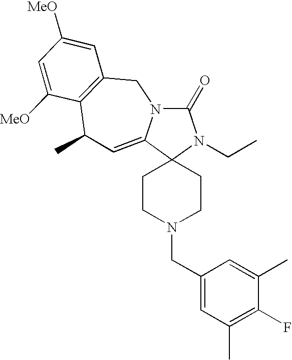 | ER-819943 | >10 |
| 54 | 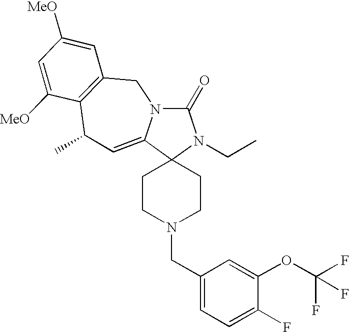 | ER-819933 | 1.15 |
| 55 | 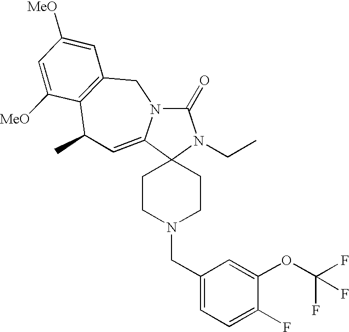 | ER-819945 | >10 |
| 56 | 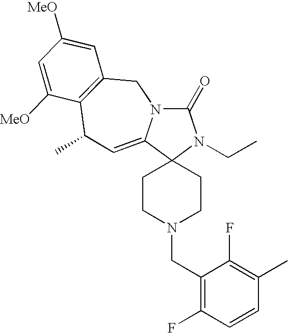 | ER-819934 | 0.10 |
| 57 | 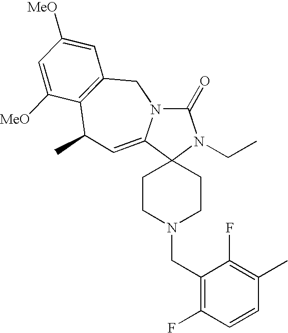 | ER-819946 | 2.97 |
| 58 | 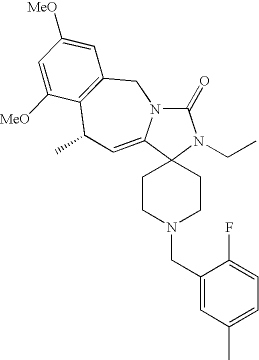 | ER-819935 | 0.13 |
| 59 | 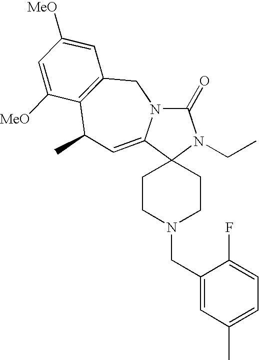 | ER-819947 | 2.6 |
| 60 | 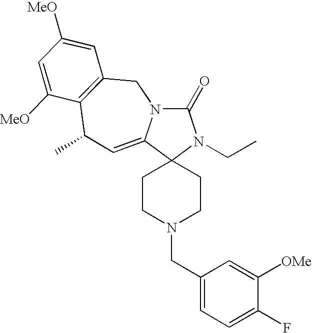 | ER-819936 | 0.12 |
| 61 | 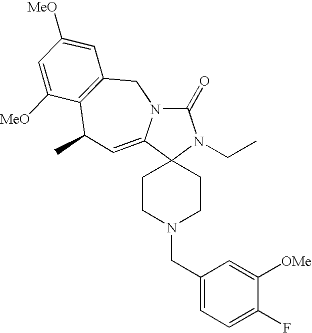 | ER-819948 | >10 |
| 62 | 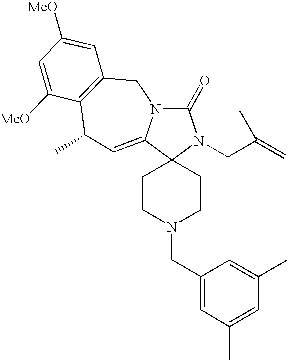 | ER-820006 | 0.06 |
| 63 | 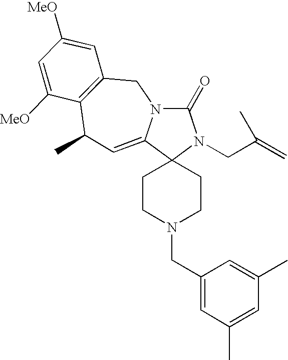 | ER-820007 | 1.26 |
| 64 | 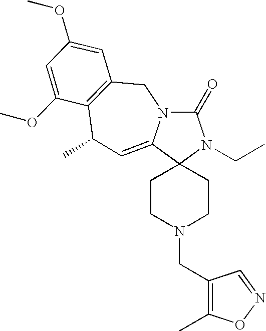 | ER-819810-01 | 0.012 |
| 65 | 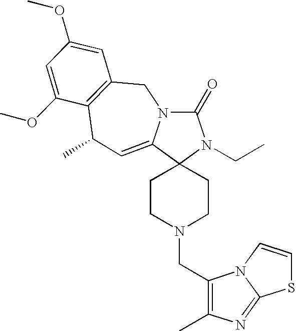 | ER-811352-02 | 0.013 |
| 66 | 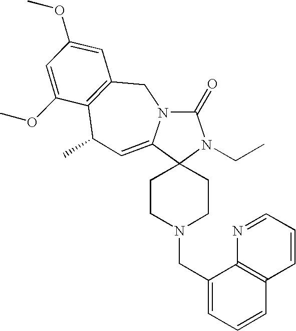 | ER-819955-01 | 0.020 |
| 67 | 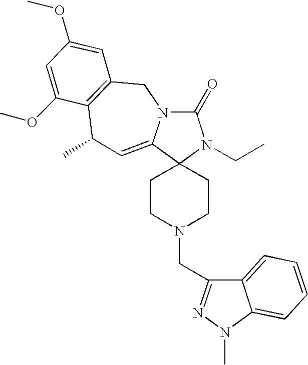 | ER-819800-01 | 0.023 |
| 68 | 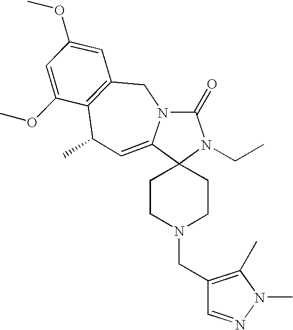 | ER-819976-01 | 0.023 |
| 69 | 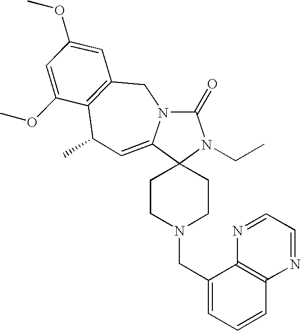 | ER-819953-01 | 0.026 |
| 70 | 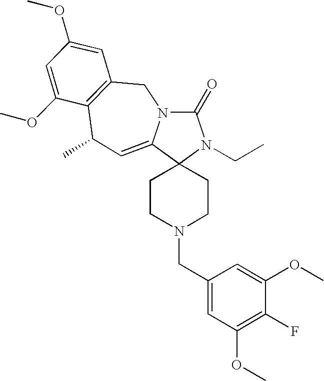 | ER-824214-00 | 0.029 |
| 71 | 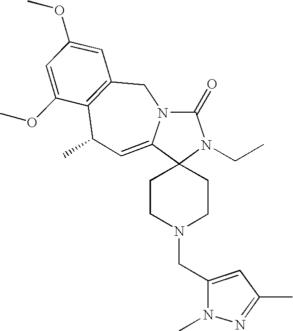 | ER-819973-01 | 0.030 |
| 72 | 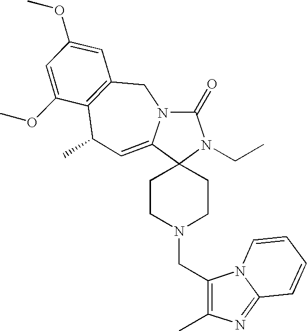 | ER-811305-01 | 0.031 |
| 73 | 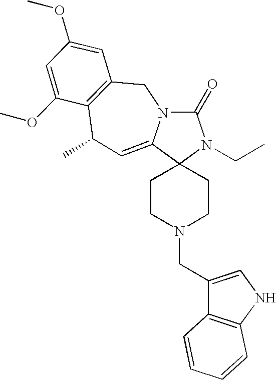 | ER-819783-01 | 0.035 |
| 74 | 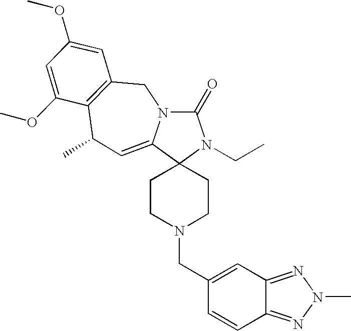 | ER-819847-01 | 0.035 |
| 75 | 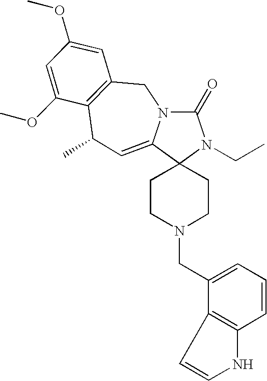 | ER-811300-01 | 0.035 |
| 76 | 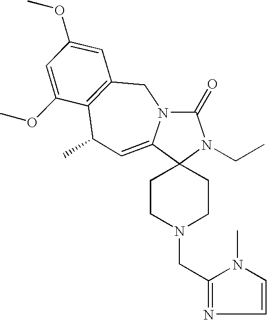 | ER-811278-01 | 0.037 |
| 77 | 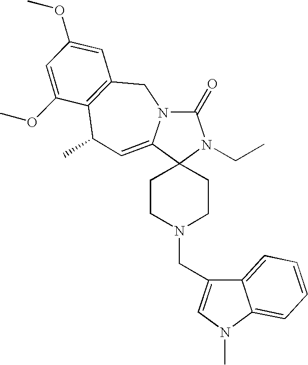 | ER-819804-01 | 0.040 |
| 78 | 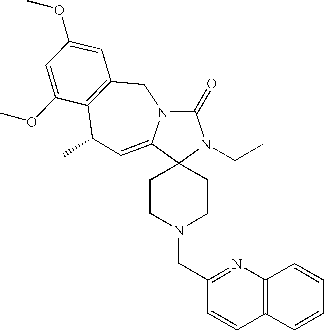 | ER-811323-01 | 0.042 |
| 79 | 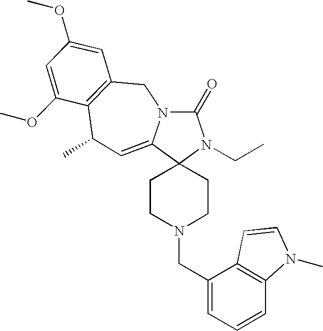 | ER-811349-01 | 0.043 |
| 80 | 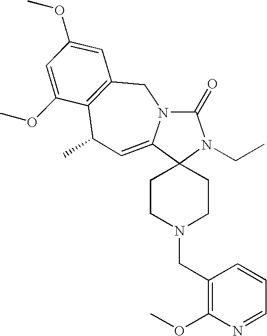 | ER-819833-01 | 0.047 |
| 81 | 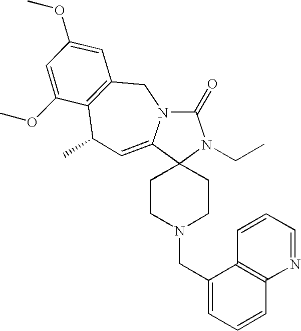 | ER-819954-01 | 0.047 |
| 82 | 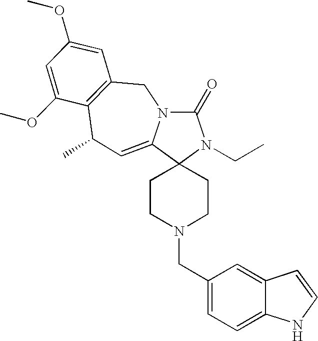 | ER-819966-01 | 0.048 |
| 83 | 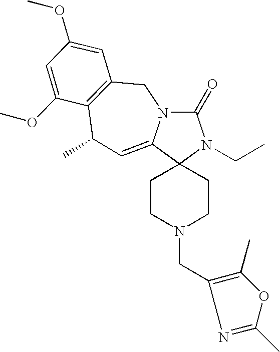 | ER-811283-01 | 0.053 |
| 84 | 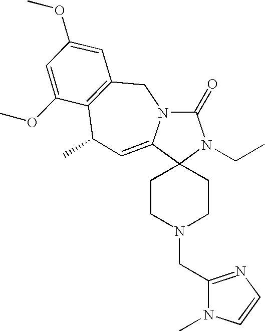 | ER-819957-01 | 0.055 |
| 85 | 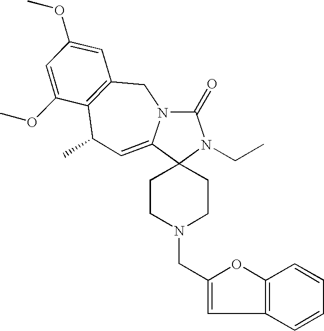 | ER-811308-01 | 0.056 |
| 86 | 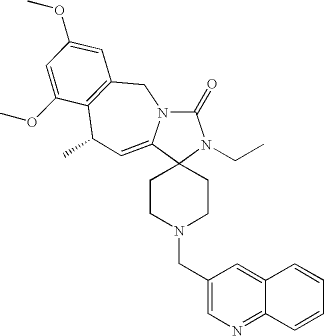 | ER-819837-01 | 0.056 |
| 87 | 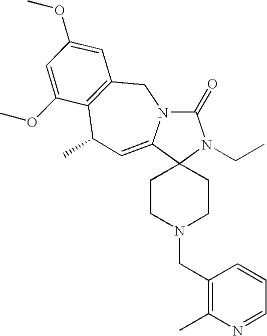 | ER-819832-01 | 0.057 |
| 88 | 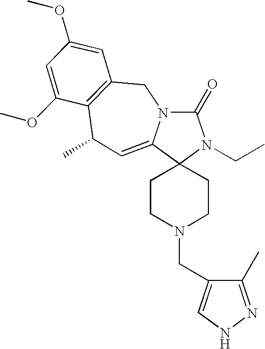 | ER-819826-01 | 0.067 |
| 89 | 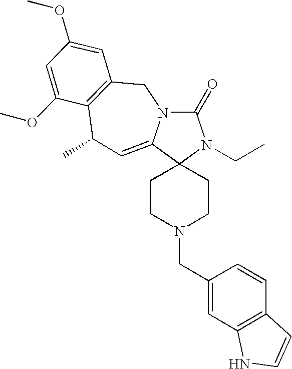 | ER-819844-01 | 0.067 |
| 90 | 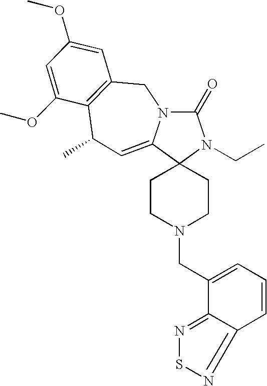 | ER-811332-01 | 0.067 |
| 91 | 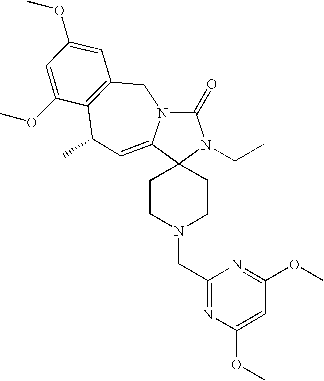 | ER-820004-01 | 0.067 |
| 92 | 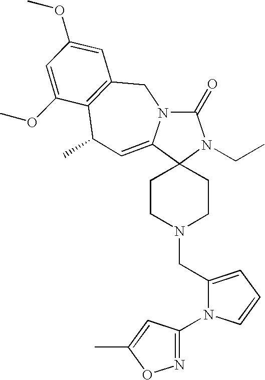 | ER-820017-01 | 0.069 |
| 93 | 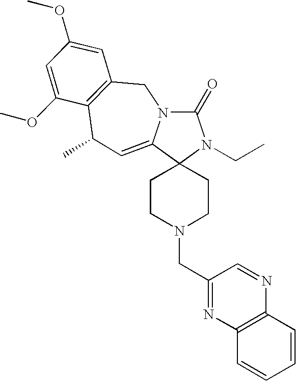 | ER-811297-01 | 0.074 |
| 94 | 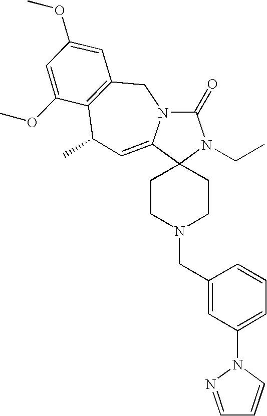 | ER-811317-01 | 0.074 |
| 95 | 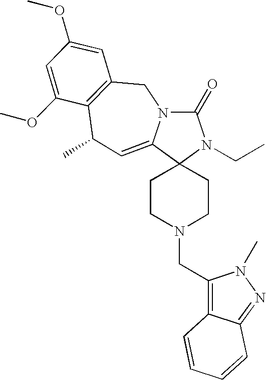 | ER-811312-01 | 0.077 |
| 96 | 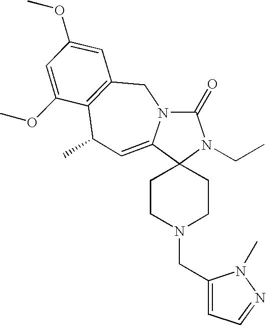 | ER-819958-01 | 0.079 |
| 97 | 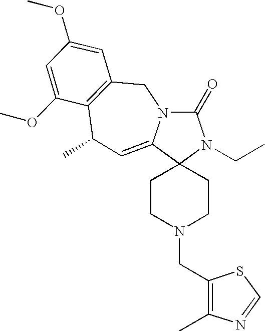 | ER-819842-01 | 0.081 |
| 98 | 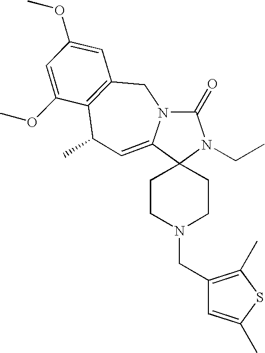 | ER-811365-01 | 0.084 |
| 99 | 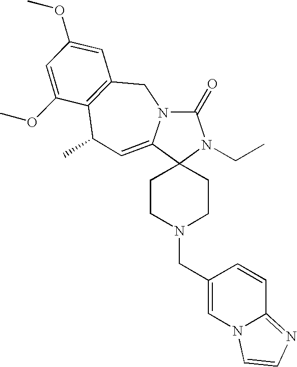 | ER-811284-01 | 0.088 |
| 100 | 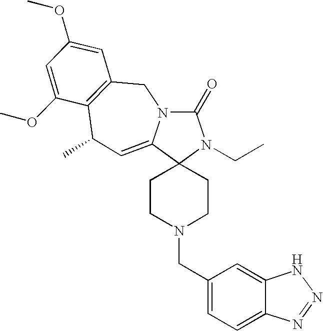 | ER-819820-01 | 0.090 |
| 101 | 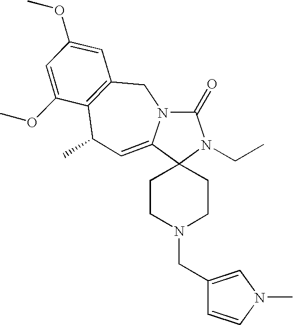 | ER-819961-01 | 0.096 |
| 102 | 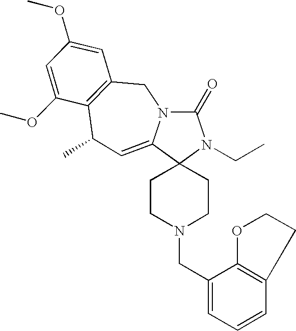 | ER-811306-01 | 0.10 |
| 103 | 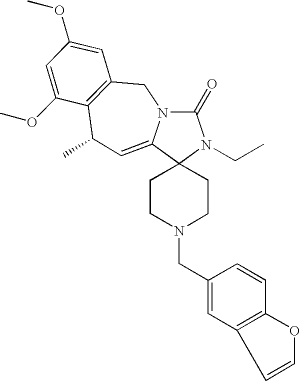 | ER-811304-01 | 0.10 |
| 104 | 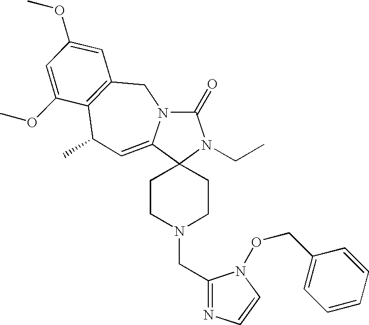 | ER-820009-00 | 0.11 |
| 105 | 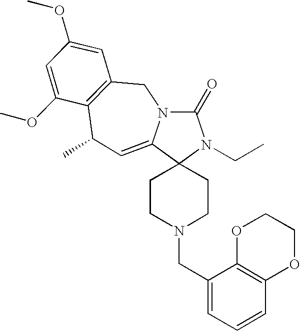 | ER-811291-01 | 0.12 |
| 106 | 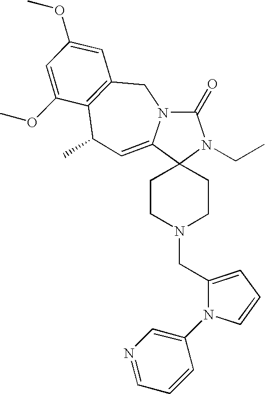 | ER-819979-01 | 0.12 |
| 107 | 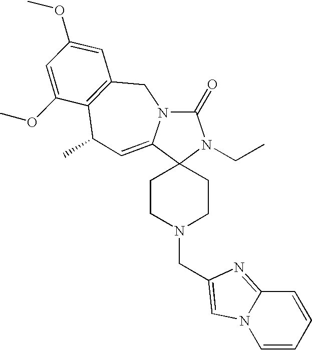 | ER-811292-01 | 0.13 |
| 108 | 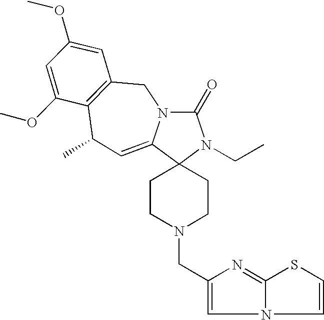 | ER-811309-01 | 0.13 |
| 109 | 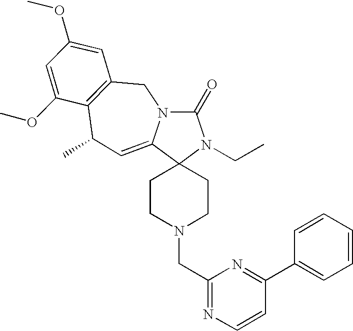 | ER-819985-01 | 0.13 |
| 110 | 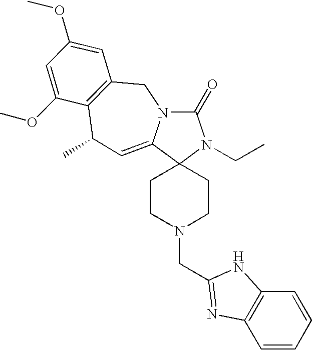 | ER-819965-01 | 0.13 |
| 111 | 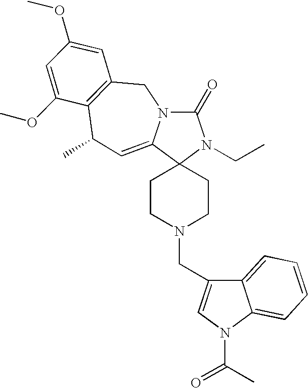 | ER-819808-01 | 0.14 |
| 112 | 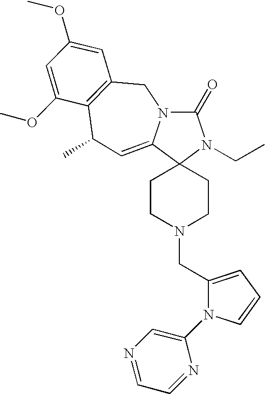 | ER-820020-02 | 0.18 |
| 113 | 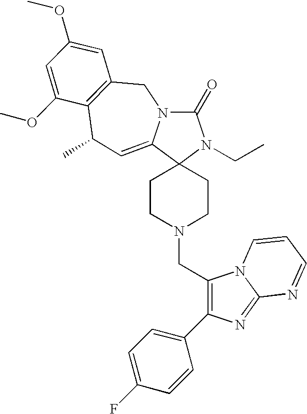 | ER-811346-02 | 0.20 |
| 114 | 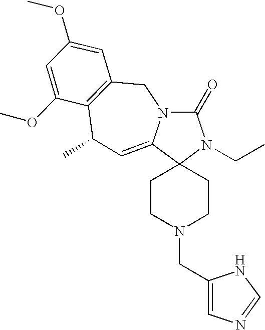 | ER-819780-01 | 0.20 |
| 115 | 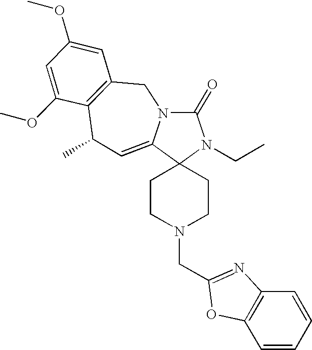 | ER-819981-01 | 0.20 |
| 116 | 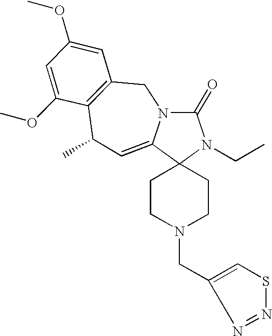 | ER-811279-01 | 0.21 |
| 117 | 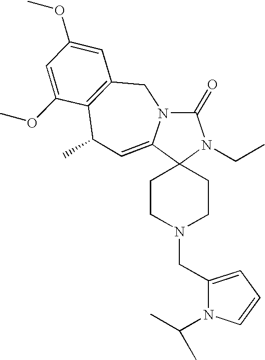 | ER-811358-01 | 0.22 |
| 118 | 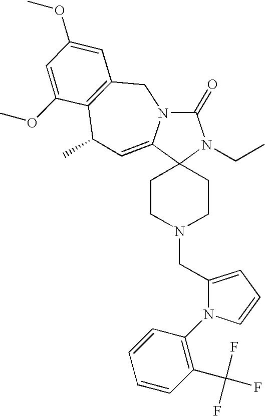 | ER-819849-01 | 0.22 |
| 119 | 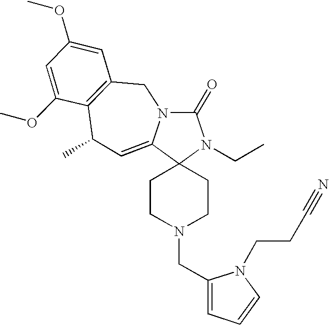 | ER-820008-00 | 0.24 |
| 120 | 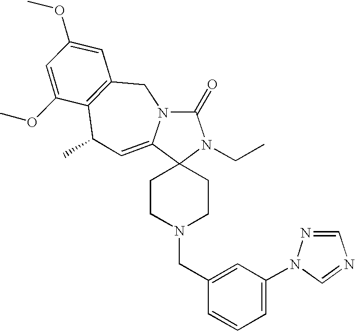 | ER-811302-01 | 0.25 |
| 121 | 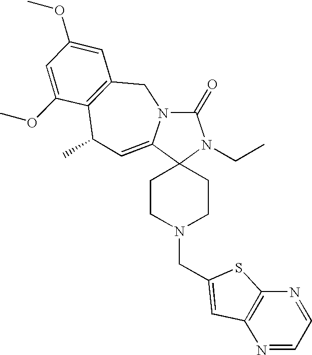 | ER-811301-01 | 0.26 |
| 122 | 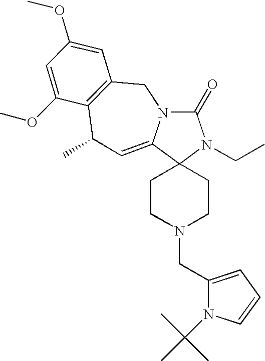 | ER-811359-01 | 0.27 |
| 123 | 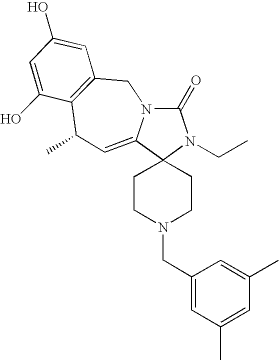 | ER-819888-00 | 0.30 |
| 124 | 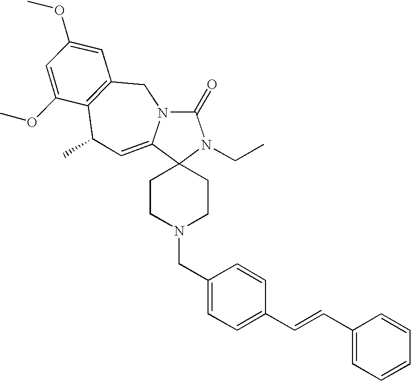 | ER-819814-01 | 0.30 |
| 125 | 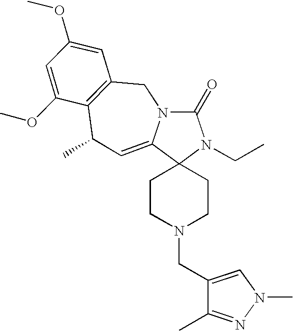 | ER-819971-01 | 0.034 |

### Example 126

### In vivo Biological Activity

### Active Immunization

Suppression of arthritis development in CIA. DBA1/J mice were immunized with bCII/CFA at day 0 then boosted at day 21 with bCII/IFA. Arthritis development was monitored over the course of study. The arthritis score is as follows: 0=normal paw, score of 1=1-2 digit inflamed paws; score of 2=3 digits or 1-2 digit+wrist or ankle inflamed, score of 3=hand+more than 2 digits inflamed; and score of 4=multiple digits (3-4)+important wrist or ankle inflammation.

(A) Partial therapeutic evaluation of compounds. Active compound was given by oral dosing once daily at the dose indicated from day 20 after induction of antibodies to collagen II but before disease development. (B) Full therapeutic evaluation of compound. Active compound was given after disease was developed (from day 7 after the second immunization). (C) X-ray analysis of mouse paws from full therapeutic CIA study. X-ray score is the index of measurement of combination of osteopenia, bone erosion and new bone formation. (D) Representative X-ray radiographs.

Data is given in Table 3 below. In general, these data compare favorably the activity of methotrexate in this model.

### Example 127

### In Vivo Biological Activity

### Passive Immunization

Suppression of arthritis development in CAIA. BALB/c mice were injected i.v. with 1 mg of anti-type II collagen antibody at day 0, and 3 days later 25 μg of LPS was injected i.p. with active compound and methotrexate (MTX) was given once daily PO from day 0 to day 7. Arthritis score and body weight was monitored over the course of study.

Data is given in Table 3 below. These data compare favorably to methotrexate, which is not particularly active in this model.

TABLE 3

Arthritis Model Approx ED₅₀ (mg/kg)

| | Active Immunization | Passive Immunization |
|---|---|---|
| Compound | (CIA mouse) | (CAIA mouse) |
| ER-819762-01 | 3 | 30-60 |
| ER-819924-01 | 3-10 | n.a. |
| ER-819973-01 | 3-10 | n.a. |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

We claim:

1. An enantiomerically pure compound of Formula Ia: 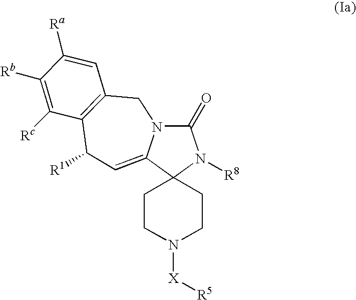
wherein:
R¹ is C₁₋₃ alkyl;
X is methylene, ethylene, propylene, ethenylene,_propenylene, or butenylene;
R⁵ is phenyl, pyrrolyl, benzimidazolyl, oxazolyl, isoxazolyl, imidazothiazolyl, quinolinyl, isoquinolinyl, indazolyl, pyridinyl, imidazopyridinyl, indolyl, benzotriazolyl, imidazolyl, benzofuranyl, benzothiadiazolyl, pyridimidinyl, benzopyranonyl, thiazolyl, thiadiazolyl, furyl, thienyl, pyrazolyl, quinoxalinyl, or naphthyl, and substituted with between 0 and 5 substituents independently selected from C₁₋₄ alkyl, C₁₋₃ alkoxy, hydroxyl, C₁₋₃ alkylthio, cyclopropyl, cyclopropylmethyl, trifluoromethoxy, 5-methylisoxazolyl, pyrazolyl, benzyloxy, acetyl, (cyanyl)C₁₋₃ alkyl, (phenyl)C₂₋₃ alkenyl; and halo;
R⁸ is H, methyl, ethyl, propyl, (C₁₋₃ alkoxy)C₁₋₃ alkyl, (C₁₋₃ alkylthio)C₁₋₃ alkyl, C₁₋₃ hydroxyalkyl, phenyl, benzyl, furyl, pyrrolyl, imidazolyl, pyrazolyl, pyrrolyl, isothiazolyl, isooxazolyl, pyridyl, and thienyl;
wherein R⁸ is substituted with between 0 and 3 substituents independently selected from methyl, ethyl, halo, hydroxyl, C₁₋₃ alkoxy, C₁₋₃ alkylthio, (C₁₋₃ alkoxy)C₁₋₃ alkyl, (C₁₋₃ alkylthio)C₁₋₃ alkyl, C₁₋₃ hydroxyalkyl, (C₁₋₃ mercaptoalkyl)phenyl, benzyl, furyl, imidazolyl, pyrazolyl, pyrrolyl, isothiazolyl, isooxazolyl, pyridyl, and thienyl; and
each of R^{a}, R^{b}, and R^{c} is independently selected from hydrogen, hydroxyl, methoxy, benzyloxy, fluoro, chloro, amino, methylamino, dimethylamino, and phenoxy;
or one pair selected from R^{a} and R^{b}, and R^{b} and R^{c}, taken together, is —O—(CH₂)—O— or —O—CH₂—CH₂—O—;
or a pharmaceutically acceptable salt, a C₁₋₆ alkyl ester or amide, or a C₂₋₆ alkenyl ester or amide thereof.

2. A compound of claim 1, wherein:
R¹ is C₁₋₂ alkyl;
R⁵ is phenyl, pyrrolyl, benzimidazolyl, oxazolyl, isoxazolyl, imidazothiazolyl, quinolinyl, isoquinolinyl, indazolyl, pyridinyl, imidazopyridinyl, indolyl, benzotriazolyl, imidazolyl, benzofuranyl, benzothiadiazolyl, pyridimidinyl, benzopyranonyl, thiazolyl, thiadiazolyl, furyl, thienyl, pyrazolyl, quinoxalinyl, or naphthyl, and substituted with between 0 and 5 substituents independently selected from C₁₋₄ alkyl, C₁₋₃ alkoxy, hydroxyl, C₁₋₃ alkylthio, cyclopropyl, cyclopropylmethyl, trifluoromethoxy, 5-methylisoxazolyl, pyrazolyl, benzyloxy, acetyl, (cyanyl)C₁₋₃ alkyl, (phenyl)C₂₋₃ alkenyl; and halo;
R⁸ is, methyl, ethyl, or propyl, wherein R⁸ is substituted with from 0 and 3 hydroxyl substituents;
X is methylene or ethylene;
R^{a} R^{b} and R^{c} are each independently selected from the group consisting of H and methoxy;
or a pharmaceutically acceptable salt thereof.

3. A compound of claim 1, wherein:
R¹ is methyl;
R⁵ is phenyl, pyrrolyl or pyrazolyl, each of which is substituted 0, 1 or 2 times with methyl;
R⁸ is ethyl;
X is methylene;
R^{a} and R^{c} are each methoxy;
R^{b} is H;
or a pharmaceutically acceptable salt thereof.

4. A compound of claim 1 selected from the group consisting of: 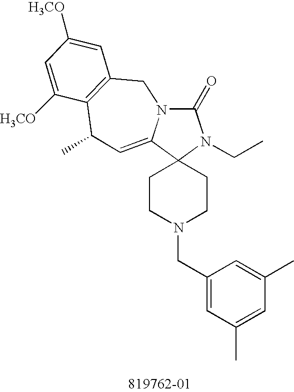
and pharmaceutically acceptable salts thereof.

5. A compound of claim 1 selected from the group consisting of: 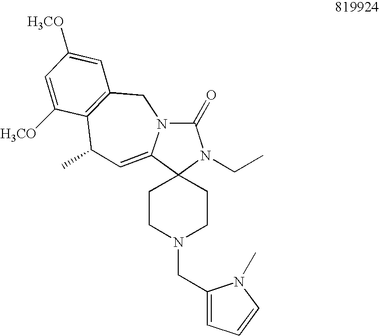
and pharmaceutically acceptable salts thereof.

6. A compound of claim 1 selected from the group consisting of: 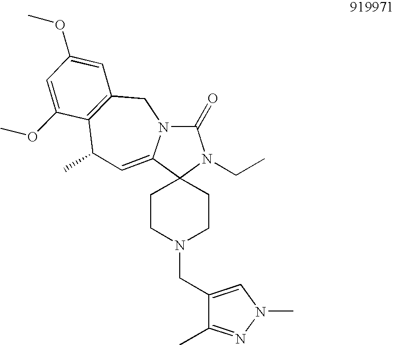
and pharmaceutically acceptable salts thereof.

7. A compound of claim 1 selected from the group consisting of: 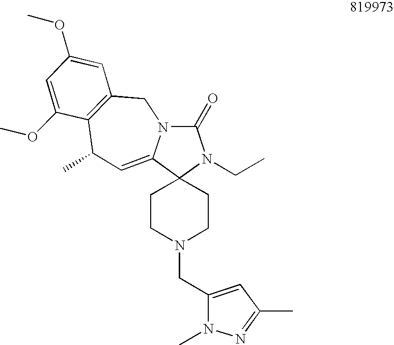
and pharmaceutically acceptable salts thereof.

8. A composition comprising a compound of claim 1 in a pharmaceutically acceptable carrier.

9. A method of treating rheumatoid arthritis in a subject in need thereof, comprising administering to said subject a compound of claim 1 in a treatment effective amount.

10. The method of claim 9, wherein:
R¹ is C₁₋₂ alkyl;
R⁵ is phenyl, pyrrolyl, benzimidazolyl, oxazolyl, isoxazolyl, imidazothiazolyl, quinolinyl, isoquinolinyl, indazolyl, pyridinyl, imidazopyridinyl, indolyl, benzotriazolyl, imidazolyl, benzofuranyl, benzothiadiazolyl, pyridimidinyl, benzopyranonyl, thiazolyl, thiadiazolyl, furyl, thienyl, pyrazolyl, quinoxalinyl, or naphthyl, and substituted with between 0 and 5 substituents independently selected from C₁₋₄ alkyl, C₁₋₃ alkoxy, hydroxyl, C₁₋₃ alkylthio, cyclopropyl, cyclopropylmethyl, trifluoromethoxy, 5-methylisoxazolyl, pyrazolyl, benzyloxy, acetyl, (cyanyl)C₁₋₃ alkyl, (phenyl)C₂₋₃ alkenyl; and halo;
R⁸ is, methyl, ethyl, or propyl, wherein R⁸ is substituted with from 0 and 3 hydroxyl substituents;
X is methylene or ethylene;
R^{a} R^{b} and R^{c} are each independently selected from the group consisting of H and methoxy;
or a pharmaceutically acceptable salt thereof.

11. the method of claim 9, said compound selected from the group consisting of: 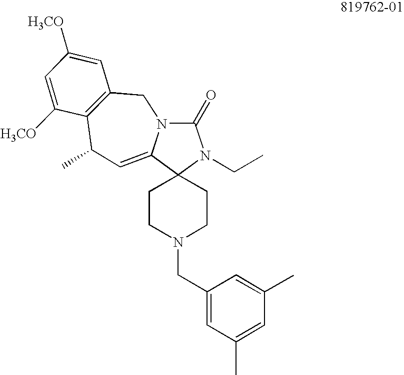
and pharmaceutically acceptable salts thereof.

12. The method of claim 9, said compound selected from the group consisting of: 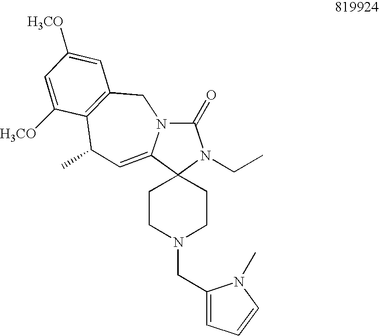
and pharmaceutically acceptable salts thereof.

13. The method of claim 9, said compound selected from the group consisting of: 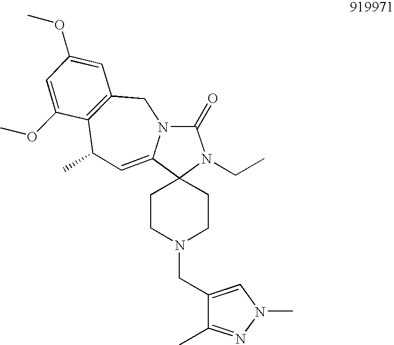
and pharmaceutically acceptable salts thereof.

14. The method of claim 9, said compound selected from the group consisting of: 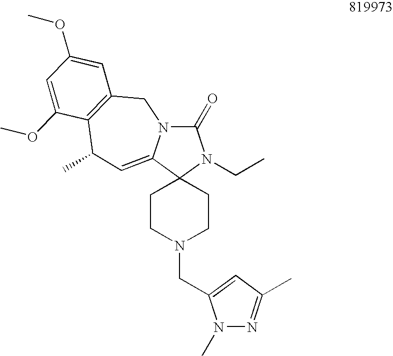
and pharmaceutically acceptable salts thereof.

15. A method of treating multiple sclerosis in a subject in need thereof, comprising administering to said subject a compound of claim 1 in a treatment effective amount.

16. The method of claim 15, wherein:
R¹ is C₁₋₂ alkyl;
R⁵ is phenyl, pyrrolyl, benzimidazolyl, oxazolyl, isoxazolyl, imidazothiazolyl, quinolinyl, isoquinolinyl, indazolyl, pyridinyl, imidazopyridinyl, indolyl, benzotriazolyl, imidazolyl, benzofuranyl, benzothiadiazolyl, pyridimidinyl, benzopyranonyl, thiazolyl, thiadiazolyl, furyl, thienyl, pyrazolyl, quinoxalinyl, or naphthyl, and substituted with between 0 and 5 substituents independently selected from C₁₋₄ alkyl, C₁₋₃ alkoxy, hydroxyl, C₁₋₃ alkylthio, cyclopropyl, cyclopropylmethyl, trifluoromethoxy, 5-methylisoxazolyl, pyrazolyl, benzyloxy, acetyl, (cyanyl)C₁₋₃ alkyl, (phenyl)C₂₋₃ alkenyl; and halo;
R⁸ is, methyl, ethyl, or propyl, wherein R⁸ is substituted with from 0 and 3 hydroxyl substituents;
X is methylene or ethylene;
R^{a} R^{b} and R^{c} are each independently selected from the group consisting of H and methoxy;
or a pharmaceutically acceptable salt thereof.

17. the method of claim 15, said compound selected from the group consisting of: 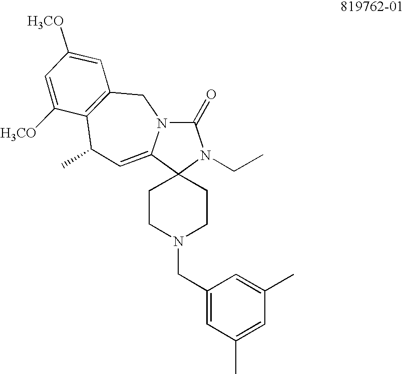
and pharmaceutically acceptable salts thereof.

18. The method of claim 15, said compound selected from the group consisting of: 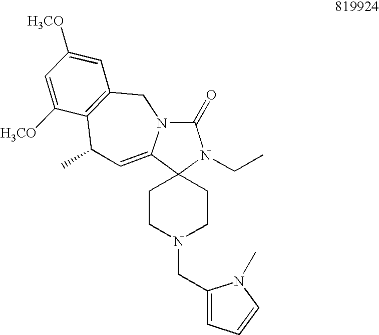
and pharmaceutically acceptable salts thereof.

20. The method of claim 15, said compound selected from the group consisting of: 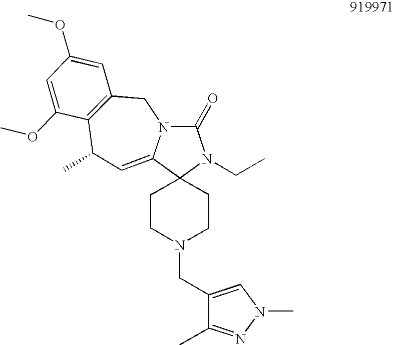
and pharmaceutically acceptable salts thereof.

21. The method of claim 15, said compound selected from the group consisting of: 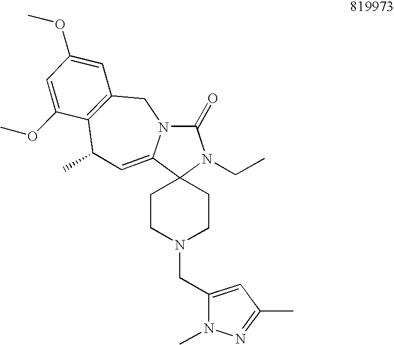
and pharmaceutically acceptable salts thereof.

22. A method of treating an autoimmune disease in a subject in need thereof, comprising administering to said subject a compound of claim 1 in a treatment effective amount;
wherein said autoimmune disease is selected from the group consisting of systemic lupus erythematosus, type 1 diabetes mellitus, psoriasis, and atherosclerosis.

23. The method of claim 22, wherein:
R¹ is C₁₋₂ alkyl;
R⁵ is phenyl, pyrrolyl, benzimidazolyl, oxazolyl, isoxazolyl, imidazothiazolyl, quinolinyl, isoquinolinyl, indazolyl, pyridinyl, imidazopyridinyl, indolyl, benzotriazolyl, imidazolyl, benzofuranyl, benzothiadiazolyl, pyridimidinyl, benzopyranonyl, thiazolyl, thiadiazolyl, furyl, thienyl, pyrazolyl, quinoxalinyl, or naphthyl, and substituted with between 0 and 5 substituents independently selected from C₁₋₄ alkyl, C₁₋₃ alkoxy, hydroxyl, C₁₋₃ alkylthio, cyclopropyl, cyclopropylmethyl, trifluoromethoxy, 5-methylisoxazolyl, pyrazolyl, benzyloxy, acetyl, (cyanyl)C₁₋₃ alkyl, (phenyl)C₂₋₃ alkenyl; and halo;
R⁸ is, methyl, ethyl, or propyl, wherein R⁸ is substituted with from 0 and 3 hydroxyl substituents;
X is methylene or ethylene;
R^{a} R^{b} and R^{c} are each independently selected from the group consisting of H and methoxy;
or a pharmaceutically acceptable salt thereof.

24. the method of claim 22, said compound selected from the group consisting of: 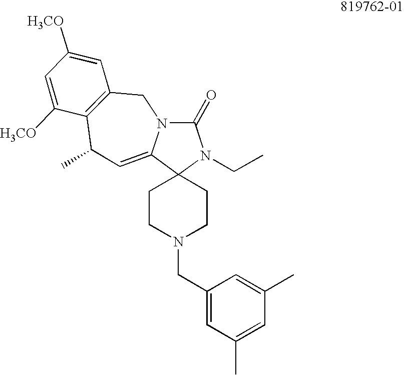
and pharmaceutically acceptable salts thereof.

25. The method of claim 22, said compound selected from the group consisting of: 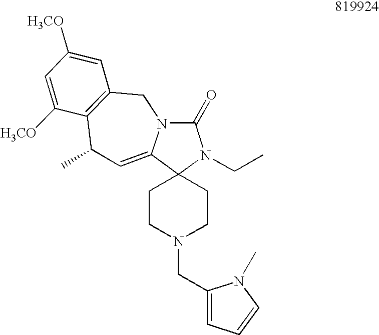
and pharmaceutically acceptable salts thereof.

26. The method of claim 22, said compound selected from the group consisting of: 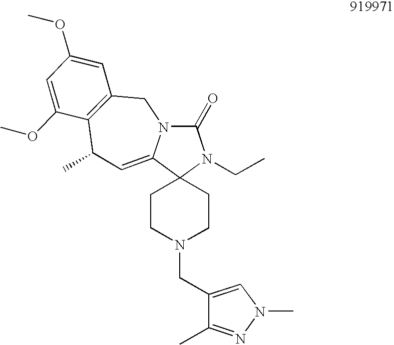
and pharmaceutically acceptable salts thereof.

27. The method of claim 22, said compound selected from the group consisting of: 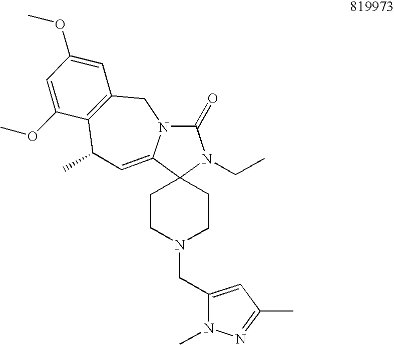
and pharmaceutically acceptable salts thereof.
